SEQUENCE LISTING (10 sequences)

SEQ ID NO: 1 - DNA, 22 bp, Artificial Sequence
  Synthetic Primer
gaagcataagtgggtggatctc

SEQ ID NO: 2 - DNA, 22 bp, Artificial Sequence
  Synthetic Primer
gtcacccaggttggaatacagt

SEQ ID NO: 3 - DNA, 22 bp, Artificial Sequence
  Synthetic Primer
catgtagaccacaaggatgcac

SEQ ID NO: 4 - DNA, 22 bp, Artificial Sequence
  Synthetic Primer
gatttcaccatgtctgtcagga

SEQ ID NO: 5 - DNA, 22 bp, Artificial Sequence
  Synthetic Primer
tttttgtagagatggggcttca

SEQ ID NO: 6 - DNA, 22 bp, Artificial Sequence
  Synthetic Primer
tgtaccaccaataaggcaacaa

SEQ ID NO: 7 - DNA, 20 bp, Artificial Sequence
  Synthetic Primer
ttcacatcccgattctgaca

SEQ ID NO: 8 - DNA, 20 bp, Artificial Sequence
  Synthetic Primer
ttgccaaatttctcctctgc

SEQ ID NO: 9 - DNA, 21 bp, Artificial Sequence
  Synthetic Primer
gaggaagggatgaatactcca

SEQ ID NO: 10 - DNA, 21 bp, Artificial Sequence
  Synthetic Primer
ctacttccttgctcgctttcc

(19) United States
(12) Patent Application Publication
(10) Pub. No.: US 2016/0017302 A1
(43) Pub. Date: Jan. 21, 2016

(54) HEPARANASE EXPRESSION IN HUMAN T LYMPHOCYTES

(71) Applicant: BAYLOR COLLEGE OF MEDICINE, Houston, TX (US)

(72) Inventors: Gianpietro Dotti, Houston, TX (US); Dario Marchetti, Seabrook, TX (US); Ignazio Caruana, Houston, TX (US)

(21) Appl. No.: 14/773,330

(22) PCT Filed: Mar. 5, 2014
(86) PCT No.: PCT/US14/20936

§ 371 Date: Sep. 5, 2015

Related U.S. Application Data
(60) Provisional application No. 61/772,591, filed on Mar. 5, 2013.

(51) Int. Cl. C12N 9/24; A61K 35/17; C12N 5/0783

(52) U.S. Cl. CPC C12N 9/2402; C12N 5/0636; A61K 35/17; C12Y 302/01166

(57) ABSTRACT

Embodiments of the present disclosure concern improvements to cell therapy for cancer. In certain embodiments, an ex vivo expanded T cell lacks endogenous heparanase expression, and amelioration of this effect allows an improvement for cancer cell therapy, including of solid tumors. In specific embodiments, ex vivo expanded T cells comprise recombinant heparanase expression.

This application claims priority to U.S. Provisional Patent Application Ser. No. 61/772,591, filed Mar. 5, 2013, which is incorporated by reference herein in its entirety.

### STATEMENT REGARDING FEDERALLY SPONSORED RESEARCH OR DEVELOPMENT

This invention was made with government support under R01CA142636-01 awarded by NCI/NIH and by PR093892 and W81XWH-10-10425 awarded by the Department of Defense. The government has certain rights in the invention.

### TECHNICAL FIELD

Embodiments of the present disclosure concern at least the fields of cell therapy, immunotherapy, molecular biology, cell biology, and medicine, including cancer medicine.

### BACKGROUND

The clinical efficacy of T-cell based therapies for cancer patients has been substantially increased by genetic modifications aimed at redirecting their antigen-specificity through the expression of chimeric antigen receptors (CARs) or ectopic α- and β-TCR chains (Pule, et al., 2008; Kalos, et al., 2011; Morgan, et al., 2006). While these tumor directed T cells have been highly effective treatment for lymphoid tumors, even in patients with significant tumor burden (Kalos, et al., 2011; Rooney, et al., 1995), their effect has generally been less striking in solid tumors such as neuroblastoma (NB) (Pule, et al., 2008), particularly when patients have large tumor burden. This limitation may in part be due to active tumor immune evasion strategies (Zou, 2005), but functional changes brought by the culture process itself may reduce tumor penetration by ex vivo cultured T cells.

CAR-engineered T-cell therapies are mostly effector and effector-memory T cells that in addition to their potent effector function (Pule, et al., 2008; Kalos, et al., 2011; Savoldo, et al., 2011), need to retain the ability to traffic and accumulate at tumor sites. Such properties involve a complex series of interactions, including the adhesion of T cells to endothelial cells and chemokine-chemokine receptor interactions, which then modulate the extravasation of T cells into antigen-rich tissues (Muller, 2003; Parish, 2006; Yadac, et al., 2003). During this process, T lymphocytes physiologically degrade the main components of the subendothelial basement membrane (BM) and of the extracellular matrix (ECM), including the heparan sulphate proteoglycans (HSPGs) that are associated with the membrane of a wide range of cells (Berfield, et al., 1999). This process of degradation of the HSPGs by T cells is also required for effective therapy of solid tumors, as the ECM is a critical component of the tumor microenvironment.

Fundamental to the degradation of ECM is the release of degradative enzymes by T cells, of which heparanase (HPSE) appears to be one of the most important. HPSE is the only known mammalian β-D-endoglycosidase capable of cleaving heparan sulphate (HS) chains of HSPGs (Parish, 2006; de Mestre, et al., 2007; Vlodaysky, et al., 2007; Yurchenco & Schittny, 1990). HPSE is first synthesized as an inactive precursor protein of ˜65 kDa, and then cleaved in two protein subunits of ˜8 and ˜50 kDa that heterodimerize to form the active HPSE protein (Vlodaysky, et al., 2007). HPSE also makes a major contribution to inflammation, and appears to be produced in large amounts by activated CD4⁺ T lymphocytes, neutrophils, monocytes and B lymphocytes (Fridman, et al., 1987; Naparstek, et al., 1984; Vlodaysky, et al., 1992). Consistent with this role in promoting tissue infiltration by T lymphocytes, HPSE plays a crucial role in experimental autoimmune encephalomyelitis (de Mestre, et al., 2007) and arthritis (Parish, 2006).

Although HPSE has been implicated in inflammation, its contribution in mediating T-cell infiltration at the tumor site remains unclear. It is also unknown which effects T cell manipulation prior to adoptive transfer would have on production of this enzyme. The present disclosure satisfies a need in the art to enhance the ability of therapeutic cells, such as ex vivo expanded cells, to be effective for cancers such as solid tumors.

### BRIEF SUMMARY

The present disclosure is directed to methods and compositions related to cell therapy. In particular embodiments, the cell therapy is for an individual in need of cell therapy, such as a mammal, including a human. The cell therapy may be suitable for any medical condition, although in specific embodiments the cell therapy is for cancer. The cancer may be of any kind, although in specific embodiments the cancer comprises one or more solid tumors in the individual; the solid tumor(s) may be benign or malignant. The individual may be of any age or either gender. In specific embodiments, the individual is known to have cancer, is at risk for having cancer, or is suspected of having cancer. The cancer may be a primary or metastatic cancer, and the cancer may be refractory to treatment. In particular embodiments, the cancer concerns treatment of solid tumors, such as breast, lung, brain, colon, kidney, prostate, pancreatic, thyroid, bone, cervical, spleen, anal, esophageal, head and neck, stomach, gall bladder, melanoma, non small cell lung cancer, lymphoma, myeloma, and so forth, for example. In alternative embodiments, the disclosure concerns treatment of non-solid tumors, such as leukemia.

Particular embodiments of the disclosure provide improvements to immunotherapy, including improvements to cell therapy. In specific embodiments, the disclosure provides improvements to adoptive T-cell based therapies. In particular aspects, the disclosure provides improvements to therapies that employ ex vivo expanded cells, such as ex vivo expanded T cells. In certain aspects, the ex vivo expanded cells are utilized for cell therapy for an individual with cancer. In particular cases, the improved ex vivo expanded cells are modified to allow the cells to be more effective than if they had not had the modification. The modified cells may be more effective for any variety of reasons, although in specific embodiments the modified cells are capable of penetrating the extracellular matrix (ECM), and also exhibit improved migration through the ECM. In certain aspects, the modified cells are able to (or are able to more effectively) degrade heparin sulphate proteoglycans (main components of ECM and cell surface). In certain aspects, the modified cells are able to (or are able to more effectively) penetrate the subendothelial basement membrane. In embodiments of the disclosure, the modified cells have a greater antitumor effect than their unmodified counterparts. In alternative embodiments, the ex vivo expanded cells are deficient in heparanase expression and the replenishment of heparanase expression allows the cells to have improved antitumor activity, although the improvement may be indirectly related or unrelated to penetration of the ECM.

In one aspect, provided herein is a composition comprising an immune cell that, in unmodified form, lacks detectable heparanase expression but that has been modified to express heparanase to detectable levels. In specific aspects, the immune cell has been manipulated ex vivo and lost endogenous expression of heparanase but is modified through recombinant technology to express heparanase, e.g., express heparanase to a degree greater than the cell's expression of heparanase prior to such genetic engineering. Thus, in one aspect, provided herein is an immune cell that has been genetically engineered to express heparanase or an active fragment thereof.

Embodiments of the disclosure provide for modified T cells that express heparanase and are effective against solid tumors, including solid tumors having abundant stroma. In specific embodiments, the modified T cells degrade the ECM of tumor stroma. In particular aspects, the modified T cells that express heparanase have an improved ability for T-cell extravasation and tumor infiltration, e.g., as compared to T cells not expressing heparanase, or expressing relatively reduced levels of heparanase.

In embodiments of the disclosure, there is an ex vivo cultured cell, comprising recombinant expression of heparanase, wherein there is no expression of endogenous heparanase in the cell or wherein existing expression of heparanase is overexpressed upon recombinant expression of heparanase. In specific aspects, the cell may lack heparanase for any reason, although in certain aspects the cell has downregulation of heparanase because of binding of a factor to the heparanase gene promoter; in certain embodiments the factor is p53. In specific embodiments, the cell is a T-cell, NK-cell, or NKT-cell. The cell may be an ex vivo expanded T-cell. The cell may be a tumor antigen-specific T cell. In certain embodiments, the immune cell, e.g., T-cell, comprises a polypeptide that targets the immune cell to a target cell expressing a particular antigen, e.g., a tumor associated antigen (TAA) or tumor specific antigen (TSA), and directs the immune cell to kill the target cell. In a specific embodiment, the polypeptide is a chimeric antigen receptor or modified T cell receptor. In another specific embodiment, the immune cell is a T cell comprising a chimeric antigen receptor (CAR), i.e., a CAR-T cell.

Embodiments of the disclosure provide pharmaceutical compositions that comprise cells that express heparanase through recombinant technology manipulation, wherein the cells would not otherwise express heparanase were it not for the recombinant technology manipulation. The pharmaceutical compositions may comprise immune cells that have undergone manipulation(s) that directly or indirectly result in loss of heparanase expression, and the cells are then modified to express heparanase. The pharmaceutical compositions may comprise carrier compositions for the cells, including at least aqueous carriers.

In embodiments of the disclosure, there is a method of improving efficacy of cell therapy, comprising the step of modifying cells for the therapy to express heparanase recombinantly. In specific embodiments, the cells lack endogenous heparanase expression and the modifying step restores heparanase expression. In certain embodiments, the cells have endogenous heparanase expression and the heparanase is overexpressed. The cells may be tumor antigen-specific T cells. The cells may be CAR-specific T cells. The cells may comprise an engineered T cell receptor or other modification aimed at improving trafficking or survival of T cells, such as chemokine receptors or cytokines.

In cases where a CAR or an engineered T cell receptor are expressed in the cells, the cells may comprise a polynucleotide (such as an expression vector) that encodes the respective CAR or engineered T cell receptor. A vector in the cells may comprise an expression construct that encodes heparanase, a CAR, an engineered T cell receptor, or a combination thereof. A single vector may comprise an expression construct that encodes heparanase, a CAR, an engineered T cell receptor, or a combination thereof, or multiple vectors may comprise expression constructs that encodes heparanase, a CAR, an engineered T cell receptor, or a combination thereof. In cases where an expression construct encodes two or more of heparanase, a CAR, and an engineered T cell receptor, their regulation of expression may be directed by the same or by different regulatory elements. In certain embodiments, the two or more of heparanase, a CAR and/or engineered T cell receptor are expressed as a single polycistronic polypeptide in which the individual polypeptides are separated by a cleavable peptide; e.g., 2A peptide. Illustrative examples of expression vectors include, but are not limited to, a plasmid or viral vector. In specific embodiments, the cell therapy is for cancer, and the cell therapy may be for a solid tumor.

In particular embodiments, a method of treating cancer (including solid tumors) in an individual is provided, comprising the step of delivering an amount of therapeutic cells to the individual therapeutically effective to treat said cancer, e.g. slow the growth of said cancer, reduce the number of tumor cells in said cancer, reduce tumor load, or eliminate said cancer, wherein the cells are ex vivo cultured cells that recombinantly express heparanase. In specific embodiments, the cells: 1) lack endogenous heparanase expression; or 2) have endogenous heparanase expression and the recombinantly expressed heparanase is overexpressed. In some cases, endogenous heparanase is engineered to increase its expression or exogenous heparanase is added to the cell. In certain embodiments, the cells may be tumor antigen-specific T cells. The cells may be CAR-specific T cells or may comprise an engineered T cell receptor. In specific embodiments, the cell therapy is for cancer, and the cell therapy may be for a solid tumor.

As described herein, heparanase production by adoptively transferred, tumor-directed T cells was studied, and it was determined whether the limited efficacy of these cells for the treatment of solid tumors results from their compromised capacity to degrade HSPGs in the tumor ECM, that in turn limits their capacity to successfully reach tumor cells within the tumor microenvironment. Thus, in certain embodiments of the disclosure, restored deficient expression of heparanase in tumor-specific T cells enhances their antitumor effects, for example in a solid tumor, such as may be shown in a suitable model, including a neuroblastoma model.

In an embodiment, there is a composition comprising ex vivo cultured immune cells that recombinantly express heparanase. In specific embodiments, the cell lacks expression of endogenous heparanase. In particular embodiments, the cell additionally endogenously expresses heparanase. In certain embodiments, expression of endogenous heparanase is upregulated compared to levels in one or more reference cells. In particular embodiments, the cell is a T-cell, NK-cell, or NKT-cell. In particular aspects, the cell is an ex vivo expanded T-cell. In some embodiments, the cell is a tumor antigen-specific T-cell. In one embodiments, the cell comprises a chimeric antigen receptor (CAR), including the cell comprising a polynucleotide encoding the CAR. In one embodiment, a polynucleotide encoding the CAR comprises an expression vector. In some cases, the expression vector comprises the polynucleotide encoding the CAR and further comprises a polynucleotide encoding heparanase. In particular embodiments, the cell comprises an engineered T cell receptor. In some cases, the cell comprises a polynucleotide encoding the engineered T cell receptor. In particular aspects, the polynucleotide encoding the engineered T cell receptor comprises an expression vector. In certain embodiments, the expression vector encodes the engineered T cell receptor and/or encodes heparanase.

In one embodiment, there is a method of treating cancer in an individual, comprising the step of delivering a therapeutically effective amount of a composition of the disclosure to the individual. In specific embodiments, the cancer comprises extracellular matrix comprising heparan sulphate proteoglycan (HSPG). In some embodiments, the cancer comprises solid tumor, and the tumor may or may not be malignant. The solid tumor may be a sarcoma, carcinoma, or lymphoma. The cells may be allogeneic to the individual or autologous to the individual. The cells may be T-cells. In some cases, the cells comprise a CAR and may comprise a polynucleotide that encodes the CAR. In some embodiments, the cells comprise an engineered T cell receptor, and the cells may comprise a polynucleotide that encodes the T cell receptor. In particular embodiments, methods of treating cancer further comprise the step of delivering one or more additional cancer therapies to the individual, such as chemotherapy, radiation, surgery, hormone therapy, and/or immunotherapy.

In one embodiment, there is a method of improving efficacy of immune cell therapy, comprising the step of modifying immune cells to recombinantly express heparanase. In specific embodiments, the cells lack expression of endogenous heparanase and the modifying step restores heparanase expression in the cells. In some embodiments, the cells additionally endogenously express heparanase. In some embodiments, the cells are tumor antigen-specific T cells. In certain embodiments, methods of improving efficacy of immune cell therapy further comprising the step of delivering the cells to an individual in need thereof. In some cases, cancer in the individual comprises extracellular matrix comprising heparan sulphate proteoglycan (HSPG). In some embodiments, the individual has a solid tumor. In particular embodiments, the cells are T cells. In particular embodiments, the cells comprise a CAR and may include a polynucleotide that encodes the CAR. In some embodiments, the cells comprise an engineered T cell receptor, and the cells may comprise a polynucleotide that encodes the T cell receptor. In particular embodiments, the modifying step comprises delivering a polynucleotide that encodes heparanase or a heparanase catalytic domain to an immune cell. In certain embodiments, the modifying step further comprises delivering a polynucleotide that encodes a CAR to the immune cell. In certain embodiments, the polynucleotide that encodes heparanase or a heparanase catalytic domain also encodes a CAR. Particular embodiments include methods wherein the modifying step further comprises delivering a polynucleotide that encodes an engineered T cell receptor to the immune cell. In some embodiments, the polynucleotide that encodes heparanase or a heparanase catalytic domain also encodes an engineered T cell receptor.

One embodiment of the disclosure includes a kit comprising any composition of the disclosure, including cells, vectors, nucleotides and, in some aspects, the kit further comprises one or more additional cancer therapeutics, such as a chemotherapy, a hormone therapy, and/or an immunotherapy.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present disclosure, reference is now made to the following descriptions taken in conjunction with the accompanying drawing, in which:

FIGS. 1A-1E demonstrates that ex vivo expanded T cells show reduced invasion of the ECM because of the loss of HPSE. Panel A. ECM invasion assay of monocytes (CD14⁺ cells, black bar), freshly isolated T lymphocytes (FT) (white bar), briefly activated T cells (BA-T) (grey bar) and ex vivo expanded T cells (LTE-T) (striped bar). Data summarize means±standard deviation (SD) of 5 independent experiments. Panel B. Western blot showing the expression of HPSE in monocytes, FT, BA-T and LTE-T CD4⁺ and CD8⁺ at different time points. β-actin staining was used to ensure equal loading of the samples. Data are from 4 donors. At day 14 of LTE-T were reactivated using OKT3/CD28 Abs and then analysed on day 15. Wild type or human HPSE transfected 293T cells were used as negative and positive controls, respectively. Panel C. Representative immunofluorescence staining for HPSE in MCF-7, monocytes, FT and LTE-T. Nuclei are stained with DAPI and shown in blue, while HPSE is stained with red-fluorescent dye (Alexa Fluor 555). Magnification is 20×. Panel D. Quantitative RT-PCR of HPSE in FT, BA-T and LTE-T CD4⁺ (black bars) and CD8⁺ (white bars). Fold change in gene expression was calculated with respect to monocytes. Data summarize means±SD of 4 independent experiments. At day 14 of culture, T cells were reactivated using OKT3/CD28 Abs, and then analysed on day 15. Panel E. HPSE enzymatic activity was assessed in supernatants collected from FT, BA-T and LTE-T CD4⁺ (circle) and CD8⁺ (square). At day 4 and 14 of culture, LTE-T were collected, washed and re-suspended in fresh media. On day 14, LTE-T were reactivated using OKT3/CD28 Abs, and analysed on day 15. For the starting time point (T=0) value, non-activated T cells rested for 48-72 hours in media were used. The tumor cell lines CHLA-255, A549 and DU-145, known to release HPSE, were used as positive controls to estimate assay sensitivity. Monocyte lysates of CD14⁺ cells pooled from 4 different donors were also used as a positive control.

FIGS. 2A-2E—LTE-T modified to express HPSE reacquire the capacity to degrade ECM. LTE-T were transduced with a retroviral vector encoding HPSE and GFP [HPSE(I)GFP]. Panel A. GFP expression of both CD4⁺ and CD8⁺ LTE-T at day 12 of culture. Panel B. qRT-PCR for HPSE in control LTE-T, HPSE(I)GFP⁺ LTE-T, human MSC (negative control), LAN-1, CHLA-255 and A549 tumor cell lines (positive controls). Data summarize the mean and SD of 3 donors. Panel C. WB showing the expression of HPSE in control and transduced LTE-T at day 12 of culture. β-actin staining was used to ensure equal loading of the samples. Panel D. ECM invasion assay of control and HPSE(I)GFP⁺ LTE-T, with or without selection based on GFP expression. Data summarize mean±SD of 9 donors. Panel E. ECM invasion assay of HPSE-transduced LTE-T in the presence or in the absence of the inhibitor, heparin H1. Data summarize mean±SD of 4 experiments.

FIGS. 3A-3G—HPSE and GD2-specific CAR co-expressed by LTE-T retain anti-GD2 specificity and have enhanced capacity to degrade ECM. LTE-T were transduced with retroviral vectors encoding either the GD2-specific CAR alone (CAR) or both the GD2-specific CAR and HPSE [CAR(I)HPSE]. Panel A. Flow cytometry analysis to detect CAR expression by control and transduced LTE-T. Panel B. WB to detect HPSE in control and transduced LTE-T. β-actin staining demonstrates equal loading of samples. Panel C. Cytotoxic activity of control, CAR⁺ and CAR⁺ HPSE⁺ LTE-T assessed by ⁵¹Cr-release assay at a 20:1 effector:target ratio. LAN-1 and CHLA-255 (GD2⁺), and Raji (GD2⁻) were used as target cells. Panel D. Transduced LTE-T release both IL-2 and IFNγ in response to GD2⁺ tumor cells. Panel E. Invasion of ECM by control, CAR⁺ and CAR⁺HPSE⁺ LTE-T. Overall data in panels C-E summarize mean±SD from 4 to 5 donors. Panels F,G. Control and transduced LTE-T were plated in the upper part of either ECM assay or insert assay, while LAN-1/GFP⁺ cells were plated in the lower chamber. After day 3 of culture, cells in the lower chamber were collected to quantify CD3⁺ T cells and GFP⁺ tumor cells by flow cytometry. Panel F illustrates representative dot plots, while Panel G summarizes mean±SD of 5 donors.

FIGS. 4A-4D provides that T cells co-expressing HPSE and GD2-CAR have enhanced antitumor activity in the presence of the ECM. Control and LTE-T transduced with retroviral vectors encoding either CAR or CAR(I)HPSE were plated in the upper part of the ECM assay and evaluated for their capacity to eliminate LAN1/GFP⁺ or CHLA-225/GFP⁺ cells plated in the lower chamber of the invasion assay. T cells and tumor cells were plated at a 15:1 ratio. After 24 hours, inserts and chambers were removed, and at day 3 of culture, invading cells were collected and stained with anti-CD3 antibody to identify T cells; GFP-expression by the tumor cells allowed these to be enumerated by flow cytometry after treatment/invasion to assess antitumor activity. The assay containing only the insert (black bars) was used to evaluate the antitumor effects of transduced T cells in the absence of ECM. Panel A and B illustrate representative dot plots of the flow cytometry analysis for culture in the presence of LAN1 and CHLA-225, respectively. Panel C and D summarize mean±SE of 5 independent experiments.

FIGS. 5A-5D—CAR-GD2⁺HPSE⁺ LTE-T show enhanced tumor infiltration in vivo and improved overall survival in two xenogenic neuroblatoma mouse models. Panel A. Kaplan-Meier analysis of mice engrafted with the tumor cell line CHLA-255 and treated with control, CAR⁺ and CAR(I)HPSE⁺ LTE-T. Panel B. Flow cytometry analysis of CD3⁺ T cells detected within the tumor samples. Dot plots are representative of 3 mice per group. Panel C. Kaplan-Meier analysis of mice engrafted with the tumor cell line LAN-1 and treated with control, CAR⁺ and CAR(I)HPSE⁺ LTE-T. Panel D. Weight of the tumors collected from euthanized mice.

FIGS. 6A-6C Re-expression of HPSE does not affect LTE-T biodistribution in vivo. CAR(I)HPSE+ and CAR+ LTE-T were labelled with the vector encoding GFP.FFluc and then infused via tail injection in NOG-SCID mice. T-cell biodistribution was evaluated by in vivo imaging at indicated time points after LTE-T infusion (Panels A). Tissues were collected from infused mice by day 12 or 19 after LTE-T infusion and stained with hematoxylin and eosin (Panels B) and anti-CD3 antibody (Panels C). 20× magnification. Human tonsil sections were used as positive control for CD3 staining.

FIGS. 7A-7B show that T-cell subsets were isolated from PBMC and stimulated.

FIGS. 8A-8B—Schematic representation of the retroviral vectors used to transduced activated T lymphocytes illustrating exemplary constructs for heparanase expression and related controls.

FIGS. 9A-9D—p53 is upregulated in LTE-T and binds to HPSE promoter. Panel A. qRT-PCR of HPSE and p53 in CD4⁺ and CD8⁺ T cells at different time points of culture. Fold change in gene expression was calculated respect to T=0. Data summarize means±SD of 3 independent experiments. Panel B. WB showing the expression of HPSE and p53 in CD3⁺ FI-T, BA-T and LTE-T. β-actin staining was used to ensure equal loading of the samples. Panel C, D. p53 ChIP in LTE-T in culture by day 14 (C), and in CD45RA⁺ cells before (T=0) and after TCR cross linking (T=72 h) (D). Input is DNA sonicated but not immunoprecipitated; IgG and p53 are DNA immunoprecipitated by the isotype and p53-specific Abs, respectively. Relative quantification was performed comparing the intensities of PCR bands of IgG and p53 to input PCR band. For this representative sample relative quantifications are: IgG 20% and p53 90% for LTE-T (C); IgG 2% and p53 4% at T=0 and IgG 53% and p53 100% at T=72 h for CD45RA⁺ cells (D).

FIGS. 10A-10D—Enhanced tumor infiltration by CAR-GD2⁺HPSE⁺ LTE-T in mice implanted with NB cells in the kidney. Panel A, B. Immunohistochemistry showing CD3⁺ T cell infiltration in tumors implanted in the kidney of mice infused with either CAR⁺ or CAR-GD2⁺HPSE⁺ LTE-T. 10× magnification (A) and 20× magnification (B). Panel C. Scatter plot of numbers of infiltrating CD3⁺ T cells per 10 high power fields in tumors collected from mice treated with either CAR⁺ or CAR(I)HPSE⁺ LTE-T. Panel D. Kaplan-Meier analysis of tumor bearing mice infused either with either CAR⁺ or CAR(I)HPSE⁺ LTE-T.

FIG. 11—Western blot showing the expression of HPSE in central-memory CD45RO+/CD62L+ (CM) and effector-memory CD45RO+/CD62L− (EM) at different time points after activation with OKT3/CD28 Abs. β-actin staining was used to ensure equal loading of the samples. Data are from a representative donor where both inactive and active HPSE forms were detectable.

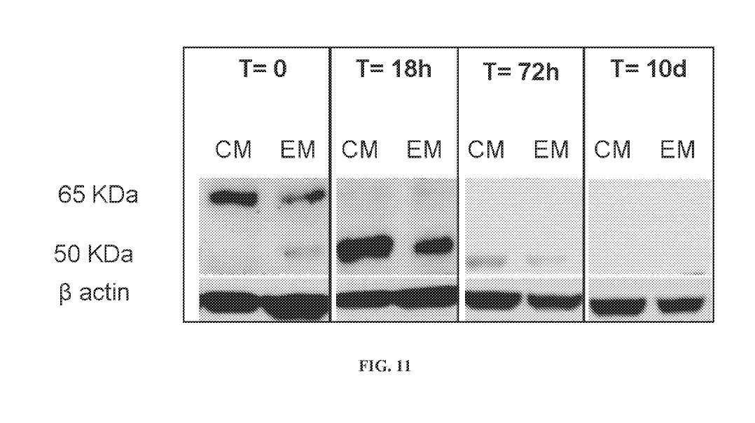

FIGS. 12A-12B—Co-expression of HPSE in GD2-specific CAR-modified LTE-T enhances antitumor activity in the presence of ECM. Panels A-B. Control and transduced LTE-T were plated in the upper part of either ECM assay or insert assay, while CHLA255/GFP+ cells were plated in the lower chamber. After day 3 of culture, cells in the lower chamber were collected to quantify CD3+ T cells and GFP+ tumor cells by flow cytometry. Panel A illustrate representative dot plots of the assay with CHLA255 GFP+ tumor cells, while Panel B summarize mean SD of 5 donors.

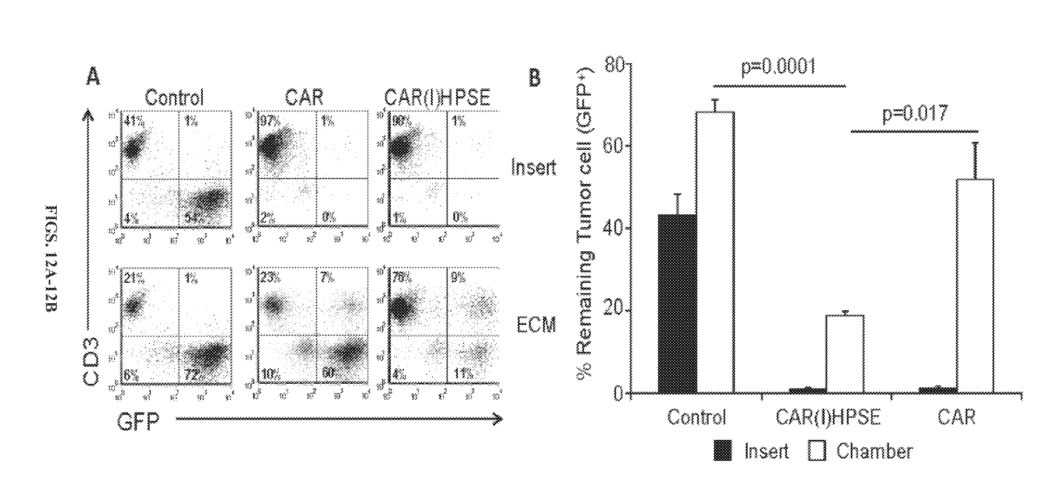

FIG. 13—The figure provides a table that summarizes the set of primers used in ChIP analysis to evaluate the p53 binding to HPSE promoter. Location of primers relatively to the origin of the promoter is also indicated. For 57-277, the sense primer comprises SEQ ID NO:1 and the antisense primer comprises SEQ ID NO:2. For 970-1167, the sense primer comprises SEQ ID NO:3 and the antisense primer comprises SEQ ID NO:4. For 1815-2030, the sense primer comprises SEQ ID NO:5 and the antisense primer comprises SEQ ID NO:6. For 2409-2687, the sense primer comprises SEQ ID NO:7 and the antisense primer comprises SEQ ID NO:8. For 2975-3274, the sense primer comprises SEQ ID NO:9 and the antisense primer comprises SEQ ID NO:10.

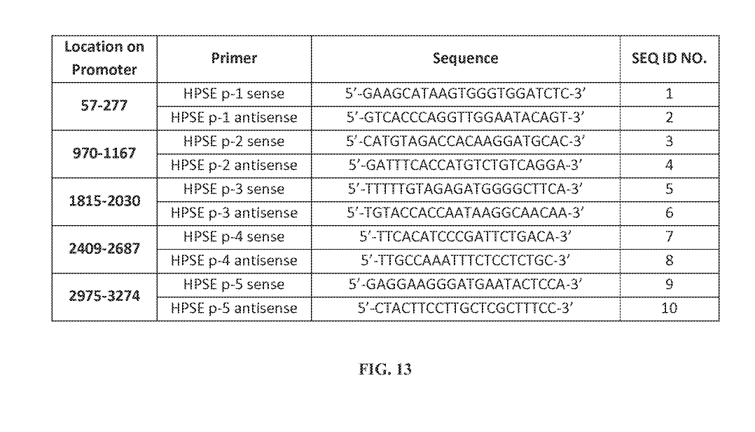

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase” consisting essentially of indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Embodiments of the disclosure address current limitations in adoptive cell transfer, particularly for cells that are not able to effectively infiltrate tumors. For example, tumor-specific T lymphocytes adoptively transferred have limited effects in patients with bulk tumors (usually more than 10 cm of the maximum diameter, in some embodiments, although the methods and compositions of the present disclosure are effective against tumors of any size). To explore this limitation, as described herein, the capacity of tumor-specific T lymphocytes manufactured for the treatment of cancer patients was characterized for their ability to degrade the extracellular matrix (ECM), which is an essential step allowing T cell extravasation. In sharp contrast with T lymphocytes isolated from the peripheral blood, cultured T lymphocytes have impaired ability to degrade the heparan sulphate proteoglycans, because they are deficient in heparanase (HPSE). Re-expression of heparanase in cultured tumor-specific T lymphocytes (for example, by gene transfer) restores their physiologic capacity to degrade the ECM, without compromising their effector function, and determines enhanced tumor T-cell infiltration and anti-tumor effects. Employing this strategy significantly enhances the activity of tumor-directed T cells in patients with solid tumors.

### I. Cells

Encompassed in the disclosure are cells that recombinantly express heparanase (for example, either by expressing exogenous heparanase or by recombinantly having an increase in expression of endogenous heparanase). In specific aspects, the cells are for adoptive transfer. The cells may be included in a pharmaceutical composition. The cells may be transformed or transfected with a vector as described herein. The recombinant heparanase-expressing cells may be produced by introducing at least one of the vectors described herein. The presence of the vector in the cell mediates the expression of a heparanase expression construct, although in some embodiments the heparanase expression construct is integrated into the genome of the cell. That is, nucleic acid molecules or vectors that are introduced into the host may either integrate into the genome of the host or it may be maintained extrachromosomally.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector. In some cases, a cell may comprise a heparanase expression construct and another expression construct, wherein the constructs are present on the same or different molecules.

Cells may comprise vectors that employ control sequences that allow them to be replicated and/or expressed in both prokaryotic and eukaryotic cells. One of skill in the art would further understand the conditions under which to incubate host cells to maintain them and to permit replication of a vector. Also understood and known are techniques and conditions that would allow large-scale production of cells of the disclosure.

In embodiments of the disclosure, there is regulation of expression of one or more of endogenous heparanase and exogenous heparanase in cells of the disclosure. The regulation of expression may include constitutive expression of heparanase, inducible expression of heparanase, environment-specific expression of heparanase, or tissue-specific expression of heparanase, and examples of such promoters are known in the art. Constitutive mammalian promoters include Simian virus 40, Immediate-early Cytomegalovirus virus, human ubiquitin C, elongation factor 1α-subunit, and Murine Phosphoglycerate Kinase-1, for example. Specific environment-specific expression of heparanse includes the use of certain regulatory elements for hypoxic conditions, for example.

In particular embodiments, the cells used in embodiments contemplated herein include eukaryotic cells, e.g., including mammalian. In certain embodiments, the cells are human, but in particular embodiments the cells are equine, bovine, murine, ovine, canine, feline, etc. for use in their respective animal. Among these species, various types of cells can be involved, such as T-cells, NK-cells, NKT-cells, etc.

The cells can be autologous cells, syngeneic cells, allogenic cells and even in some cases, xenogeneic cells with respect to the individual receiving them. The cells may be modified by changing the major histocompatibility complex (“MHC”) profile, by inactivating β₂-microglobulin to prevent the formation of functional Class I MHC molecules, inactivation of Class II molecules, providing for expression of one or more MHC molecules, enhancing or inactivating cytotoxic capabilities by enhancing or inhibiting the expression of genes associated with the cytotoxic activity, or the like.

In some instances specific clones or oligoclonal cells may be of interest, where the cells have a particular specificity, such as T cells and B cells having a specific antigen specificity or homing target site specificity.

The exemplary T-cells may be modified in a way other than recombinantly expressing heparanase. For example, one may wish to introduce genes encoding one or both chains of a T-cell receptor. For example, in addition to providing for expression of a gene having therapeutic value such as heparanase and, optionally, another therapeutic gene, in some embodiments the cell is modified to direct the cell to a particular site. The site can include anatomical sites, and in particular embodiments includes solid tumors. An increase in the localized concentrations of the cells can be achieved following their enhanced capability to migrate through the ECM (because of the heparanase expression) by expressing surface membrane proteins on the host cell that will enable it to bind to a target site such as a naturally occurring epitope on a target cell. There are numerous situations where one would wish to direct cells to a particular site, where release of a therapeutic product could be of great value or where pathways in the cell are triggered that directly or indirectly result in apoptosis of the cell.

In one embodiment, the host cell is a T cell comprising recombinant heparanase but also comprising an engineered TCR receptor, engager molecule, and/or a CAR, for example. Naturally occurring T cell receptors comprise two subunits, an α-subunit and a β-subunit, each of which is a unique protein produced by recombination event in each T cell's genome. Libraries of TCRs may be screened for their selectivity to particular target antigens. An “engineered TCR” refers to a natural TCR, which has a high-avidity and reactivity toward target antigens that is selected, cloned, and/or subsequently introduced into a population of T cells used for adoptive immunotherapy. In contrast to engineered TCRs, CARs are engineered to bind target antigens in an MHC-independent manner. In particular embodiments, a CAR comprises an extracellular binding domain including, but not limited to, an antibody or antigen binding fragment thereof; a transmembrane domain; one or more intracellular costimulatory signaling domains and a primary signaling domain.

In specific embodiments, an immune cell of the disclosure is subject to upregulation of expression of endogenous heparanase. The level of expression of endogenous heparanase may be upregulated compared to levels in a reference cell or cells. Reference cells may be cells that lack exogenous heparanase, unmodified immune cells, and so forth. The level of expression of endogenous heparanase may be increased by one or more means, including by incorporating a strong promoter in the genomic regulatory elements of the endogenous heparanase of the cell. In some cases, one can engineer the cell to express one or more transcription factors that turn on expression of endogenous heparanase.

In various embodiments, a T-cell comprises increased heparanase and one or more polynucleotides encoding engager molecules that recognize the same target antigen as a CAR or engineered TCR expressed by the T-cell. In particular embodiments, a CAR or engineered TCR expressing T-cell comprises one or more polynucleotides encoding engager molecules that recognize a target antigen that is different than the target antigen recognized by a CAR or engineered TCR, but that is expressed on the same target cell. Embodiments of the disclosure provide a polynucleotide sequence that encodes an engager molecule, e.g., an engager polypeptide. Such engager polypeptides generally comprise an antigen recognition domain and an activation domain. The engager molecule's antigen recognition domain may be designed so as to bind to one or more molecules present on target cells, while engager molecule's activation domain binds to a molecule present on effector cells, such as T lymphocytes, for example. Once the engager molecule's activation domain has bound effector cells, the activation domain can activate the effector cells. In certain embodiments, when the activation domain of the engager binds to the activation molecule on the immune cell, and the antigen recognition domain binds to the target-cell antigen, the immune cell kills the target cell. In certain embodiments, the engager is a protein, e.g., an engineered protein. In specific embodiments, the activation domain of the engager is or comprises an antibody or an antigen-binding fragment or portion thereof, e.g., a single chain variable fragment (scFv). On other specific embodiments, the antigen recognition domain is or comprises an antibody or an antibody fragment or an antigen-binding fragment or portion thereof, e.g., a monoclonal antibody, Fv, or an scFv, or it may comprise ligands, peptides, soluble T-cell receptors, or combinations thereof. In certain embodiments, the activation domain and antigen recognition domain are joined by a linker, e.g., a peptide linker. The activation domain of an engager molecule can provide activation to immune cells. The skilled artisan recognizes that immune cells have different activating receptors. For example CD3 is an activating receptor on T-cells, whereas CD16, NKG2D, or NKp30 are activating receptors on NK cells, and CD3 or an invariant TCR are the activating receptors on NKT-cells. Engager molecules that activate T-cells may therefore have a different activation domain than engager molecules that activate NK cells. In specific embodiments, e.g., wherein the immune cell is a T-cell, the activation molecule is one or more of CD3, e.g., CD3γ, CD3δ or CD3ε; or CD27, CD28, CD40, CD134, CD137, and CD278. In other specific embodiments, e.g., wherein the immune cell is a NK cell, the activation molecule is CD16, NKG2D, or NKp30, or wherein the immune cell is a NKT-cell, the activation molecule is CD3 or an invariant TCR. In certain other embodiments, the engager additionally comprises one or more other domains, e.g., one or more of a cytokine, a costimulatory domain, a domain that inhibits negative regulatory molecules of T-cell activation, or a combination thereof. In specific embodiments, the cytokine is IL-15, IL-2, and/or IL-7. In other specific embodiments, the co-stimulatory domain is CD27, CD80, CD83, CD86, CD134, or CD137. In other specific embodiments, the domain that inhibits negative regulatory molecules of T-cell activation is PD-1, PD-L1, CTLA4, or B7-H4.

Cells of the disclosure harboring an exogenous molecule(s) for expression of heparanase or intended to harbor same may also comprise a CAR (which generally comprises a tumor-associated antigen (TAA)-binding domain (most commonly a scFv derived from the antigen-binding region of a monoclonal antibody), an extracellular spacer/hinge region, a transmembrane domain and an intracellular signaling domain). The CAR may be first generation, second generation, or third generation (CAR in which signaling is provided by CD3ζ together with co-stimulation provided by one or more of CD28 and a tumor necrosis factor receptor (TNFr), such as 4-1BB or OX40), for example. The CAR may be specific for EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracelluar matrix of tumors, such as oncofetal variants of fibronectin, tenascin, or necrotic regions of tumors. The CAR (by way of example only) and heparanase may be on the same or different vectors. In some cases the CAR also comprises one or more cytokines (such as IL-2, IL-7, or IL-15, for example). Chimeric antigen structure and nomenclature is known in the art, e.g., see U.S. Pat. Nos. 7,741,465; 5,906,936; 5,843,728; 6,319,494; 7,446,190; 5,686,281; 8,399,645; and U.S. Patent Application Publication Nos. 2012/0148552, the disclosures of each of which are incorporated herein by reference in their entireties.

In many situations, it may be desirable to kill the modified cells, such as when the object is to terminate the treatment, the cells become neoplastic, in research where the absence of the cells after their presence is of interest, and/or another event. For this purpose one can provide for the expression of certain gene products in which one can kill the modified cells under controlled conditions, such as a suicide gene. Suicide genes are known in the art, e.g., the iCaspase9 system in which a modified form of caspase 9 is dimerizable with a small molecule, e.g., AP1903. See, e.g., Straathof et al., Blood 105:4247-4254 (2005).

### II. Therapeutic Uses of the Cells

An embodiment of the disclosure relates to the use of modified cells as described herein for the prevention, treatment or amelioration of a cancerous disease, such as a tumorous disease. In particular, the pharmaceutical composition of the present disclosure may be particularly useful in preventing, ameliorating and/or treating cancers in which having heparanase renders the cells of the pharmaceutical composition more effective than if the cells lacked heparanase. In specific embodiments, cancer cells being treated with pharmaceutical compositions are effectively treated because cells of the pharmaceutical compositions express heparanase that degrades the ECM of the cancer cells. In particular embodiments, the cancer is in the form of a solid tumor.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

An individual may be subjected to compositions or methods of the disclosure that is at risk for a solid tumor. The individual may be at risk because of having one or more known risk factors, such as family or personal history, being a smoker, having one or more genetic markers, and so forth.

Possible indications for administration of the composition(s) of the heparanase-expressing immune cells are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervix cancer and kidney cancer, lung cancer, gastric cancer, cancer of the small intestine, liver cancer, pancreas cancer, gall bladder cancer, cancers of the bile duct, esophagus cancer, cancer of the salivary glands and cancer of the thyroid gland. In particular embodiments, the administration of the composition(s) of the disclosure is useful for all stages and types of cancer, including for minimal residual disease, early cancer, advanced cancer, and/or metastatic cancer and/or refractory cancer, for example.

The disclosure further encompasses co-administration protocols with other compounds that are effective against cancer. The clinical regimen for co-administration of the inventive cell(s) may encompass co-administration at the same time, before, or after the administration of the other component. Particular combination therapies include chemotherapy, radiation, surgery, hormone therapy, or other types of immunotherapy.

By way of illustration, cancer patients or patients susceptible to cancer or suspected of having cancer may be treated as follows. Cells modified as described herein may be administered to the patient and retained for extended periods of time. The individual may receive one or more administrations of the cells. Illustrative cells include ex vivo expanded T-cells. The cell would be modified at least to express an active part or all of heparanase and is provided to the individual in need thereof. The cells may be injected directly into the tumor, in some cases. An exemplary heparanase nucleotide sequence is in GenBank® Accession No. NM_{—}006665, and an exemplary heparanase polypeptide sequence is in GenBank® Accession No. NP_{—}006656, both of which are incorporated by reference herein in their entirety. An active part or all of the entire sequence may be incorporated into the cell, although in specific aspects the part of heparanase that is incorporated includes any domain required for enzyme activity, for example.

In some embodiments, the genetically modified cells are encapsulated to inhibit immune recognition and are placed at the site of the tumor. For example, the cells may be encapsulated in liposomes, alginate, or platelet-rich plasma.

Another embodiment includes modification of antigen-specific T-cells with heparanase, where one can activate expression of a protein product to activate the cells. The T-cell receptor could be directed against tumor cells, pathogens, cells mediating autoimmunity, and the like. By providing for activation of the cells, for example, an interleukin such as IL-2, one could provide for expansion of the modified T cells in response to a ligand. Other uses of the modified T-cells would include expression of homing receptors for directing the T-cells to specific sites, where cytotoxicity, upregulation of a surface membrane protein of target cells, e.g. endothelial cells, or other biological event would be desired.

In another embodiment, antigen-specific T cells may be modified to export hormones or factors that are exocytosed. By providing for enhanced exocytosis, a greater amount of the hormone or factor will be exported; in addition, if there is a feedback mechanism based on the amount of the hormone or factor in the cytoplasm, increased production of the hormone or factor will result. In one aspect, one may provide for induced expression of the hormone or factor, so that expression and export may be induced concomitantly.

III. Introduction of Constructs into Cells

The heparanase constructs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagensis, etc. as appropriate. The construct(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In specific embodiments, heparanase is introduced into the cells as an RNA for transient expression. RNA can be delivered to the immune cells of the disclosure by various means including microinjection, electroporation, and lipid-mediated transfection, for example. In particular aspects, introduction of constructs into cells may occur via transposons. An example of a synthetic transposon for use is the Sleeping Beauty transposon that comprises an expression cassette including the heparanase gene of active fragment thereof.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, 1987; Mansour, et al., 1988; and Joyner, et al., 1989.

The constructs may be introduced as a single DNA molecule encoding at least heparanase and optionally another gene, or different DNA molecules having one or more genes. The constructs may be introduced simultaneously or consecutively, each with the same or different markers. In an illustrative example, one construct would contain heparanase under the control of particular regulatory sequences.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

### IV. Administration of Cells

The cells that have been modified to express heparanase (such as with DNA constructs) may be grown in culture under selective conditions, and cells that are selected as having the construct may then be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g. a mammal, in a wide variety of ways. The cells are introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

In particular embodiments, the route of administration may be intravenous, intraarterial, intraperitoneal, or subcutaneous, for example. Multiple administrations may be by the same route or by different routes.

Determination of appropriate dose levels are routinely performed in the art. In specific embodiments, the following regimen may be employed: dose level 1: 2×10⁷/m²; dose level 2: 1×10⁸/m²; dose level 3: 2×10⁸/m² based on transduced T cells.

The DNA introduction need not result in integration in every case. In some situations, transient maintenance of the DNA introduced may be sufficient. In this way, one could have a short-term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described herein at least in part.

In particular cases, a plurality of immune cells of the disclosure are delivered to an individual with cancer. In specific embodiments, a single administration is required. In other embodiments, a plurality of administration of cells is required. For example, following a first administration of the engineered immune cells, there may be examination of the individual for the presence or absence of the cancer or for a reduction in the number and/or size of tumors, for example. In the event that the cancer shows a need for further treatment, such as upon tumor growth after the first administration, an additional one or more deliveries of the same engineered immune cells (or, optionally, another type of cancer therapy, including another type of immunotherapy, and/or chemotherapy, surgery and/or radiation) is given to the individual. In some cases, a reduction of tumor size in an individual indicates that the particular immunotherapy is effective, so further administrations of same are provided to the individual.

It should be appreciated that the system is subject to variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

### V. Nucleic Acid-Based Expression Systems

In aspects of the disclosure, there are cells that express heparanase, wherein the heparanase expression is produced from recombinant DNA in the cells. The heparanase coding sequence may be provided on a vector, including an expression vector, for example. Other gene products (such as a CAR and/or an engineered T cell receptor and/or engager molecule) may be expressed from the same expression vector, or they may be present in a cell on separate vector(s) from the heparanase.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of a promoter, one positions the 5” end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In specific embodiments, the heparanase expression is under control of an inducible or tissue-specific promoter. Tissue-specific promoters are known in the art, but in specific embodiments the tissue-specificity is tailored to the tissue in which the cancer is located. The identity of tissue-specific promoters or elements, as well as assays to characterize their activity, is well known to those of skill in the art, such as hypoxia-inducible promoters.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the disclosure, the use of internal ribosome entry sites (IRES) elements are used to create multigene, or polycistronic, messages, and these may be used in the disclosure.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

C. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

D. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Components of the present disclosure may be a viral vector that encodes heparanase. Non-limiting examples of virus vectors that may be used to deliver a nucleic acid of the present disclosure are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the cells of the present disclosure as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell-lines (Miller, 1992).

In order to construct a heparanase retroviral vector, a nucleic acid (e.g., one encoding part or all of heparanase) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present disclosure. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

E. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

F. Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

G. Ex vivo Transformation

Methods for tranfecting eukaryotic cells and tissues removed from an organism in an ex vivo setting are known to those of skill in the art. Thus, it is contemplated that cells or tissues may be removed and transfected ex vivo using heparanase or other nucleic acids of the present disclosure. In particular aspects, the transplanted cells or tissues may be placed into an organism. In preferred facets, a nucleic acid is expressed in the transplanted cells.

### VI. Kits

Any of the compositions described herein may be comprised in a kit. In a non-limiting example, one or more cells for use in cell therapy that harbors recombinantly expressed heparanase and/or the reagents to generate one or more cells for use in cell therapy that harbors recombinantly expressed heparanase may be comprised in a kit. The kit components are provided in suitable container means. In specific embodiments, the kits comprise recombinant engineering reagents, such as vectors, primers, enzymes (restriction enzymes, ligase, polymerases, etc.), buffers, nucleotides, etc.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the disclosure, cells that are to be used for cell therapy are provided in a kit, and in some cases the cells are essentially the sole component of the kit. The kit may comprise instead or in addition reagents and materials to make the cell recombinant for heparanase. In specific embodiments, the reagents and materials include primers for amplifying heparanase, nucleotides, suitable buffers or buffer reagents, salt, and so forth, and in some cases the reagents include vectors and/or DNA that encodes heparanase and/or regulatory elements therefor.

In particular embodiments, there are one or more apparatuses in the kit suitable for extracting one or more samples from an individual. The apparatus may be a syringe, scalpel, and so forth.

In some cases of the disclosure, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

In some cases of the disclosure, the cell in the kit may be modified to express a therapeutic molecule other than heparanase. The other therapeutic molecule may be of any kind, but in specific embodiments, the therapeutic molecule is a chimeric antigen receptor, for example.

In some cases, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### VII. Combination Therapy

In certain embodiments of the disclosure, methods of the present disclosure for clinical aspects are combined with other agents effective in the treatment of hyperproliferative disease, such as anti-cancer agents (which may also be referred to as a cancer therapy). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present disclosure, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent treatment by intervals ranging from minutes to weeks. In embodiments where the other agent and present disclosure are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, present disclosure is “A” and the secondary agent, such as radio- or chemotherapy, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer., or any analog or derivative variant of the foregoing. In specific embodiments, chemotherapy is employed in conjunction with the disclosure, for example before, during and/or after administration of the disclosure. Exemplary chemotherapeutic agents include at least dacarbazine (also termed DTIC), temozolimide, paclitaxel, cisplatin, carmustine, fotemustine, vindesine, vincristine, or bleomycin.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155, and the like.

Immunotherapy may include interleukin-2 (IL-2) or interferon (IFN), for example. In certain embodiments, the immunotherapy is an antibody against a Notch pathway ligand or receptor, e.g., an antibody against DLL4, Notch1, Notch2/3, Fzd7, or Wnt. In certain other embodiments, the immunotherapy is an antibody against r-spondin (RSPO) 1, RSPO2, RSPO3 or RSPO4.

D. Genes

In yet another embodiment, the secondary treatment is a gene therapy in which a therapeutic polynucleotide is administered before, after, or at the same time as the clinical embodiments of the present disclosure. A variety of expression products are encompassed within the disclosure, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present disclosure to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present disclosure by establishment of an autocrine or paracrine effect on hyperproliferative cells. Increased intercellular signaling by elevation of the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Restored Expression of Heparanase in Tumor Specific T Cells Enhances their Antitumor Effects in a Neuroblastoma Model

Adoptive T-cell based therapies have shown promising results in patients with lymphomas and other hematological malignancies, but appear less effective in solid tumors. Specifically, recent clinical trial in neuroblastoma (NB) showed antitumor efficacy by CAR-modified T cells only in patients with modest bulk disease, suggesting that ex vivo expanded effector T cells may have limited capacity to penetrate and migrate through the extracellular matrix (ECM) of solid tumors.

Although the mechanisms that regulate migration of activated and effector T cells through the ECM have not been extensively investigated, the inventors found that, unlike circulating T cells, ex vivo expanded T cells lack the expression of heparanase (HPSE), a crucial enzyme involved in the degradation of the heparan sulphate proteoglycans (HSPGs), which compose the subendothelial basement membrane (BM) and ECM. Importantly, the lack of this enzymatic activity paralleled with a significantly impaired invasion capacity of ex vivo expanded T cells (8%±6%) as compared to resting T cells (23%±8%) or shortly activated T cells (34%±8%) (p=0.01). The inventors therefore evaluated whether HPSE restoration in ex vivo cultured antigen-specific T cells by retroviral gene modification rescued their invasion properties, resulting in an improved antitumor activity. When HPSE and a GD2-specific CAR (CAR) were co-expressed in T cells to target NB, CAR(I)HPSE+ T cells retained phenotypic characteristics and antitumor activity comparable to CAR+ T cells, but acquired superior capacity to invade the ECM (66%±1%) as compared to CAR+ T cells (13%±9%; p<0.001). Importantly, the in vitro antitumor activity of CAR(I)HPSE+ T cells was improved as compared to CAR+ T cells when co-culture experiments with NB tumor cells were performed in the presence of ECM (residual tumor cells: 68%±3%, 52%±9% and 19%±1% for control, CAR+ and CAR(I)HPSE+ T cells, respectively) (p=0.0001 control vs. CAR(I)HPSE and p=0.017 CAR vs. CAR(I)HPSE). When antitumor activity was compared in a xenograft neuroblastoma mouse model, mice treated with CAR(I)HPSE+ T cells had a significantly improved survival by day 40 as compared with mice treated with control T cells (p<0.001) or CAR+ T cells (p<0.007). In addition, 47% of the mice infused with CAR(I)HPSE+ were tumor free by day 40 compared with 28% of mice infused with CAR+ T cells. CAR(I)HPSE+ T treated mice showed greater infiltration of the tumor by T cells (4.6%±2.4%), as compared to tumors collected from mice treated with control (0.6%±0.5; p=0.029) or CAR+ T cells (0.1%±0.1; p=0.043) In conclusion, the restored expression of HPSE in antigen-specific T lymphocytes has a significant impact for T-cell immunotherapy of solid tumors.

### Example 2

### Restoring Deficient Expression of Heparanase in Tumor-Specific T Lymphocytes Enhances their Anti-Tumor Effects

Expanded T Cells have Impaired Capacity to Degrade the ECM Due to the Lack of HPSE.

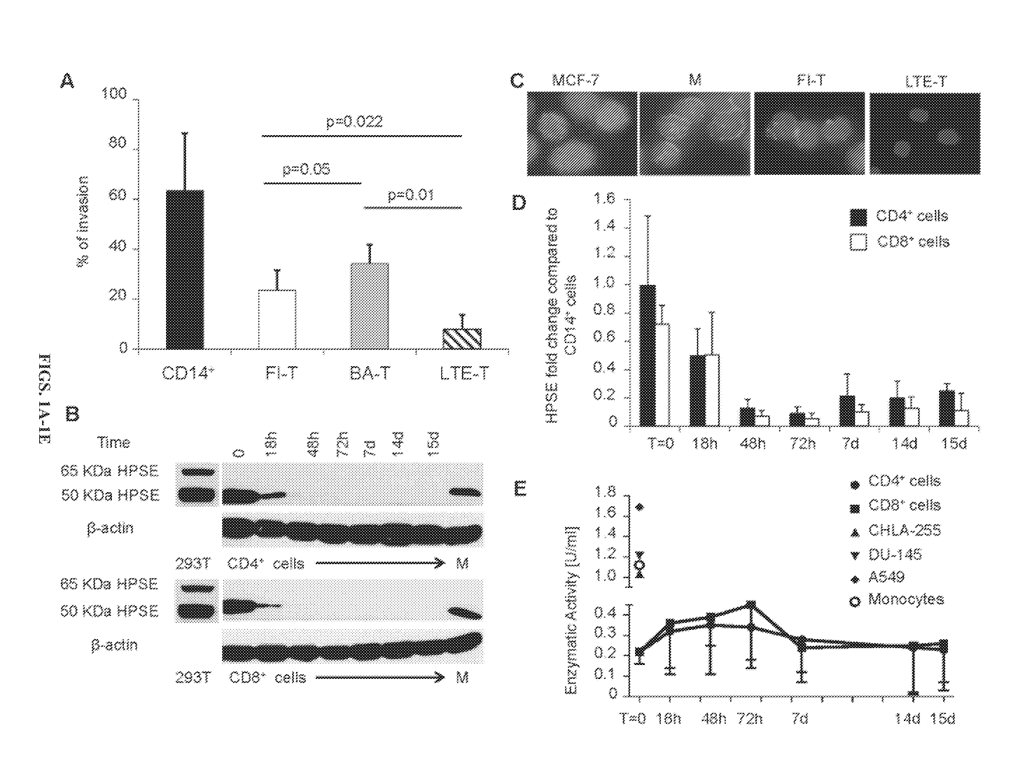

It was first assessed whether ex vivo expanded T cells were defective in their capacity to degrade the ECM. Using a Matrigel-based Invasion assay, they compared freshly isolated resting T cells (FT) (naive, effector-memory and central-memory T cells), briefly activated T cells (BA-T) exposed for 24 hours to OKT3/CD28 Abs, long term ex vivo expanded T cells (LTE-T) (activated and cultured for 12-14 days) (central-memory and effector-memory T cells), and freshly isolated monocytes (positive control). As expected, monocytes isolated from 5 different healthy donors showed the highest capacity to degrade the ECM (63%±23%) (FIG. 1A). Consistent with previously reported data in rodents (de Mestre, et al., 2007), BA-T showed enhanced invasion of the ECM as compared to FT (34%±8% versus 23%±8%, respectively; p=0.05). LTE-T, however, had significantly reduced ability to degrade the ECM (8%±6%) as compared to both BA-T (p=0.01) and FT (p=0.022).

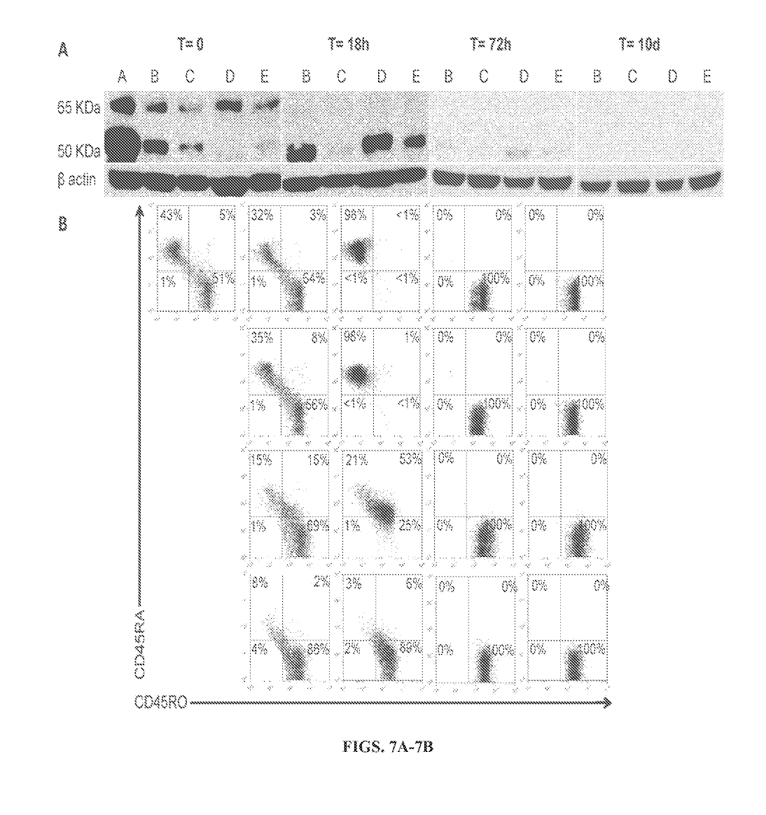

To dissect the mechanisms responsible for the above observations, the expression and function of HPSE was evaluated in each cell group. Consistent with the invasion assay, monocytes and both CD4⁺ and CD8⁺ FT and BA-T retained expression of the active form of HPSE (50 KDa), while LTE-T lost expression of HPSE by day 2 of culture, and remained consistently negative during the culture period (FIG. 1B). Furthermore, HPSE was not re-expressed even when LTE-T were rested and then reactivated using OKT3/CD28 Abs on day 14 of culture. The lack of HPSE expression by LTE-T was confirmed by immunofluorescence (FIG. 1C). By separating central-memory (CD45RO⁺CD62L⁺) and effector-memory (CD45RO⁺CD62L⁻) cells from peripheral blood, the transition from the latent (65 kDa) to the active (50 kDa) form of HPSE was also demonstrated in both subsets 18 hours after stimulation with OKT3/CD28 Abs, and the subsequent permanent loss of the enzyme (FIG. 7)

The absence of HPSE protein in LTE-T was associated with the down-regulation of the HPSE mRNA, as assessed by quantitative RT-PCR. As shown in FIG. 1D, HPSE-specific mRNA decreased immediately after activation in both CD4⁺ and CD8⁺ T cells, and remained low over the 14 day culture period as compared to CD14⁺ cells (p<0.005 for CD4⁺ and p<0.031 for CD8⁺ T cells). Re-activation of LTE-T by day 14 of culture with OKT3/CD28 Abs did not induce up-regulation of HPSE mRNA. This lack of cellular HSPE in LTE-T was also confirmed by the lack of enzymatic activity in the culture supernatant. As shown in FIG. 1E, HPSE enzymatic activity was detected in supernatants collected within the first 72 hours after activation of FT which can be attributed to accumulation in the culture media, but the enzymatic activity returns to background levels after 72 hours (from 0.34 U/ml±0.2 U/ml and 0.45 U/ml±0.27 U/ml, for CD4⁺ and CD8⁺ respectively, to 0.22 U/ml±0.06) (FIG. 1E).

Tumor Suppressor p53 Regulates HPSE Gene Expression by Binding to its Promoter.

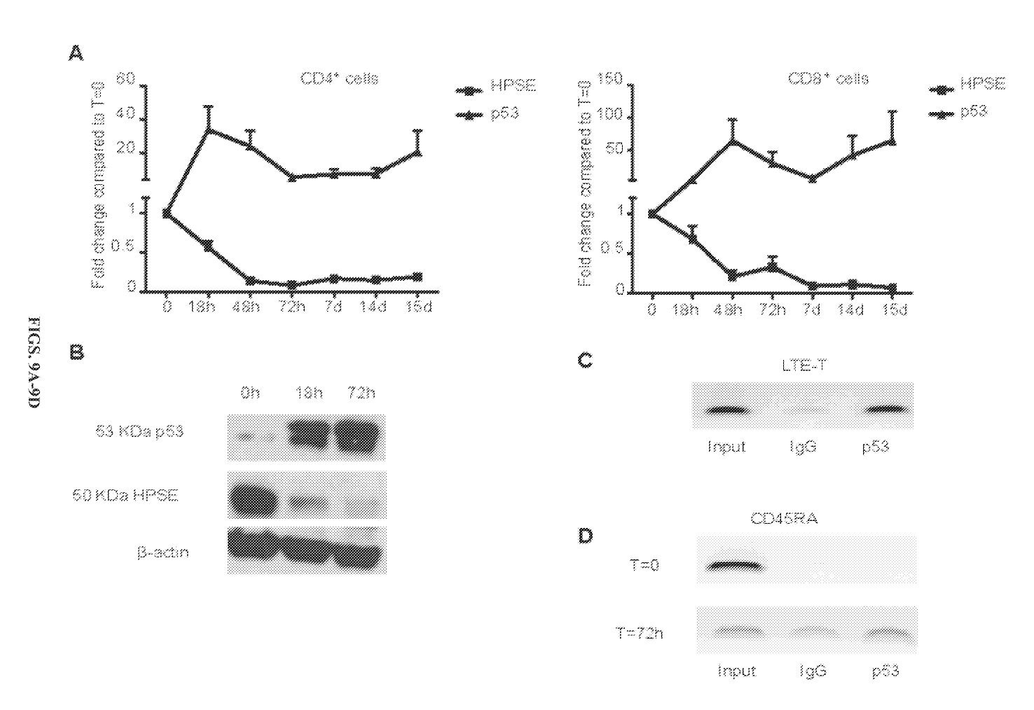

Based on previous studies showing that p53 is down-regulated or mutated with loss of function in tumor cells that over-express HPSE, it was considered that functional p53 in activated T lymphocytes would be involved in the down-regulation of the HPSE mRNA. It was found that p53 mRNA (FIG. 9A) and protein subunits (FIG. 9B) were persistently upregulated in both CD4⁺ and CD8⁺ T cells upon activation, and during ex vivo culture. P53 upregulation and HPSE down-regulation were indeed linked since p53 bound to HPSE promoter in LTE-T as assessed by p53 chromatin immunoprecipitation (ChIP) (FIG. 9C). To further demonstrate that this event was not simply observed in T cells cultured ex vivo (LTE-T), but physiologically occurs during the transition from naïve (CD45RA⁺) to antigen-experienced T cells (CD45RO⁺), p53 ChIP was repeated in freshly isolated CD45RA⁺ T cells before and after activation by T-cell receptor (TCR) cross linking. As shown in FIG. 9D, naive CD45RA⁺ T cells showed p53 binding to HPSE promoter only 72 hours after TCR cross linking, which dictates their transition from naïve to antigen-experienced T cells. Thus p53 upregulation in activated T cells contributes in permanently down-regulating HPSE mRNA expression.

Re-Expression of HPSE Restores the Capacity of Ex Vivo Expanded LTE-T to Degrade ECM.

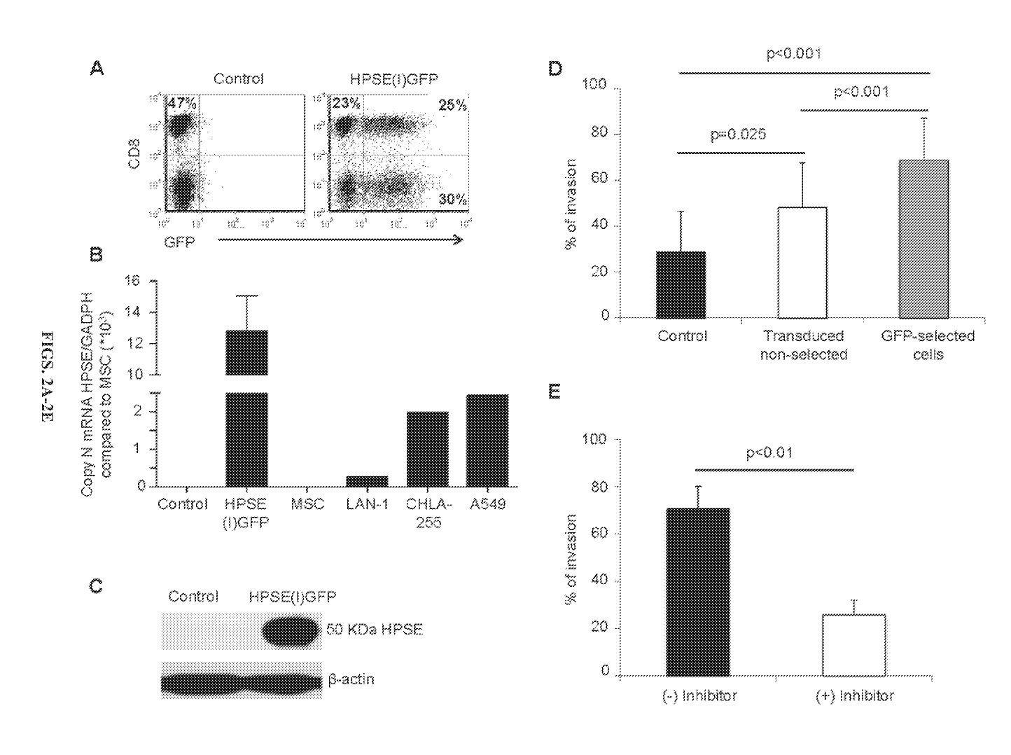

Having found that LTE-T down-regulates the expression of HPSE, thereby losing their capability to degrade ECM, it was considered that HPSE re-expression in LTE-T through retroviral gene transfer would restore their invasion capability. As illustrated in FIG. 2A, LTE-T transduced with a retroviral vector encoding both GFP and HPSE expressed GFP (51%±18%) and HPSE as assessed by qRT-PCR and WB (FIGS. 2B and C). As demonstrated in functional assays, HPSE(I)GFP⁺ LTE-T better degraded ECM (48%±19%) than control LTE-T (29%±18%; p=0.025) (FIG. 2D). This difference was further strengthened when transduced T cells were enriched for HPSE expression based upon selection of GFP positive cells (>90%), and before being tested by the Matrigel™ cell invasion assay (69%±19%, p<0.001). The addition of the HPSE-inhibitor, Heparin H1 (Nakajima, et al., 1984), confirmed that the restored invasion properties of HPSE(I)GFP⁺ LTE-T were HPSE-specific, as the invasion of GFP-sorted LTE-T was significantly reduced from 74%±14% to 29%±9% (p<0.01) (FIG. 2E). Thus re-expression of HPSE in LTE-T cultured ex vivo for adoptive T-cell transfer restores the physiologic property of memory cells to degrade ECM.

Co-Expression of HPSE in GD2-Specific CAR-Modified T Lymphocytes Enhances Invasion of the ECM without Compromising Effector Function.

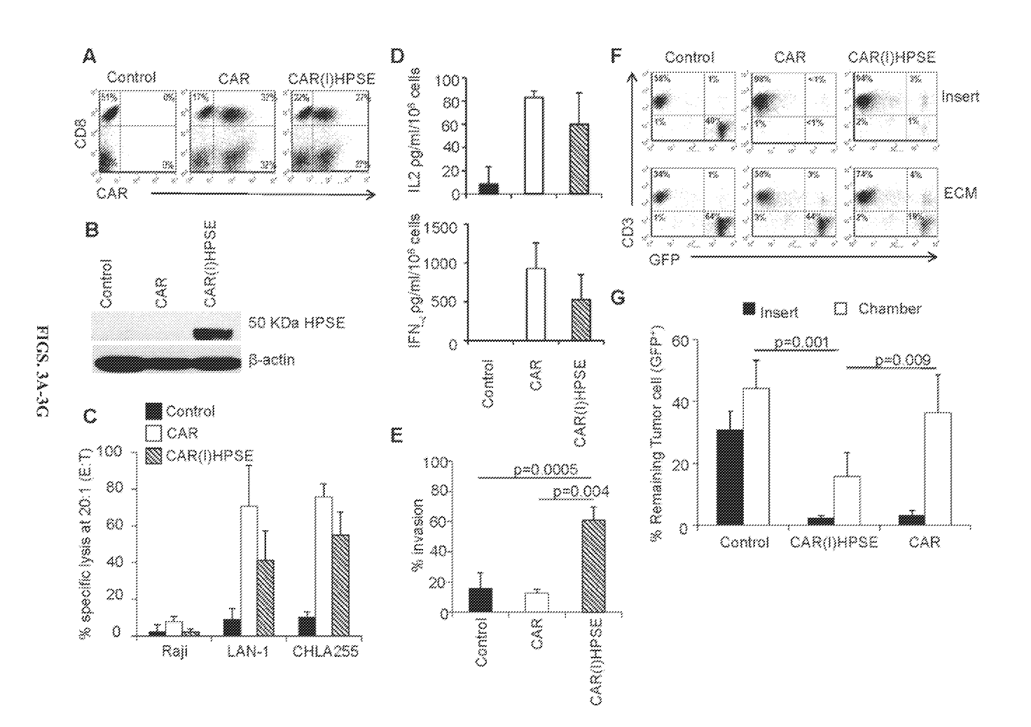

Having demonstrated that expressing HPSE in LTE-T restores their capacity to degrade the ECM, it was next determined whether this property could be coupled with an antitumor specificity. NB was used as a model, and T cells were generated targeting the NB-associated antigen GD2 by the expression of a GD2-specific CAR (Pule, et al., 2005). LTE-T from 5 healthy donors were transduced with retroviral vectors encoding either the CAR alone or both HPSE and CAR (CAR(I)HPSE). On day 14 of culture, CAR expression was 71%±14% and 56%±6% when CAR and CAR(I)HPSE vectors were used respectively (FIG. 3A). CAR molecules were expressed by both CD4⁺ and CD8⁺ T cells (39%±19% and 60%±18%, CD4⁺ and CD8⁺ respectively; CAR(I)HPSE: 38%±13% and 61%±13%, CD4⁺ and CD8⁺ respectively). HPSE was consistently detected by western blot in T cells transduced with the CAR(I)HPSE vector (FIG. 3B). The CAR(I)HPSE⁺ LTE-T retained effector function against NB target cells. In a standard ⁵¹Cr-release assay, both CAR⁺ and CAR(I)HPSE⁺ LTE-T specifically lysed GD2⁺ LAN1 cells (with a killing at a 20:1 E:T ratio of 71%±22% and 41%±16%, respectively) and GD2⁺ CHLA-255 cells (76%±7% and 55%±13%, respectively). CAR and CAR(I)HPSE LTE-T showed negligible activity against the GD2⁻ target cell line Raji (8%±3% and 2%±2%, respectively) (FIG. 3C). As expected, control LTE-T lysed none of these targets. The antitumor activity of CAR-modified T cells was associated with a preserved Th1 cytokine profile with retained release of IFNγ (927±328 and 527±320 ρg/ml/10⁶ cells for CAR⁺ and CAR(I)HPSE⁺ LTE-T, respectively) and IL-2 (83±6 and 61±27 ρg/ml/10⁶ cells CAR⁺ and CAR(I)HPSE⁺ LTE-T, respectively) (FIG. 3D). In sharp contrast to their comparable cytotoxic function, only CAR(I)HPSE⁺ LTE-T degraded ECM significantly well (66%±1%) compared to CAR⁺ or control LTE-T (13%±9% and 16%±10%, respectively) (p=0.004 and p<0.001) (FIG. 3E). To prove ex vivo that LTE-T co-expressing HPSE and CAR have increased antitumor activity in presence of ECM, LTE-T and tumor cells were plated in a Matrigel cell invasion assay in which LTE-T must degrade ECM to reach and eliminate the tumor targets. After 3 days of culture, both CAR⁺ and CAR(I)HPSE⁺ LTE-T eliminated LAN-1 tumor cells equally well in the absence of ECM (insert) (<3% residual GFP tumor cells) compared to control LTE-T (31%±6% residual GFP LAN-1 cells) (FIG. 3F,G). By contrast, in the presence of ECM, CAR(I)HPSE⁺ LTE-T eliminated all but 16%±8% of LAN-1 cells compared to residual 37%±12% in the presence of CAR⁺ LTE-T (p=0.001) (FIG. 3F,G). Control LTE-T did not show antitumor activity in any condition (either insert or ECM) (residual GFP LAN-1 45%±9%). Identical results were obtained with the NB line CHLA-255. Thus only LTE-T co-expressing HPSE and CAR show robust antitumor activity in presence of ECM.

T Cells Co-Expressing HPSE and the GD2-Specific CAR have Enhanced Antitumor Activity in the Presence of the ECM.

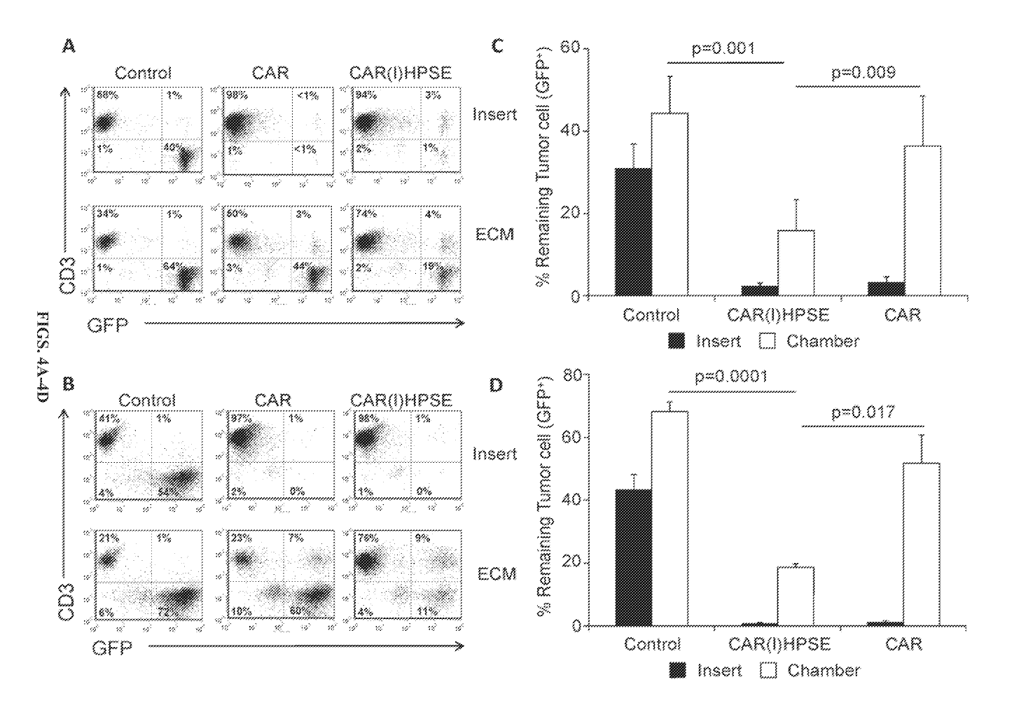

It was next determined if co-expression of the HPSE and GD2-specific CAR enhanced anti-NB activity in the presence of the ECM. They plated LTE-T and tumor cells in a Matrigel Invasion assay and measured the capacity of T cells to degrade the ECM and then target CHLA-255 and LAN1 tumor cells expressing GFP (for quantification). As illustrated in FIGS. 4A and 4B, after 3 days of culture both CAR⁺ and CAR(I)HPSE⁺ LTE-T eliminated LAN1 and CHLA-255 tumor cells equally well in the absence of ECM (less than <3% residual GFP cells), as compared to control LTE-T (31%±6% and 42%±10% of residual GFP cells, respectively). By contrast, in the presence of the ECM, CAR(I)HPSE⁺ LTE-T eliminated all but 16%±8% and 19%±1% of LAN1 and CHAL-255 cells, respectively as compared to residual 37%±12% and 52%±9% in the presence of CAR⁺ LTE-T (p=0.001). As expected control LTE-T did not show antitumor activity in any condition (residual LAN1 and CHAL-255 45%±9% and 68%±3%, respectively). FIGS. 4C and 4D summarize the mean SD.

T Cells Co-Expressing HPSE and CAR-GD2 Improve Overall Survival in a Xenograft Mouse Model of NB.

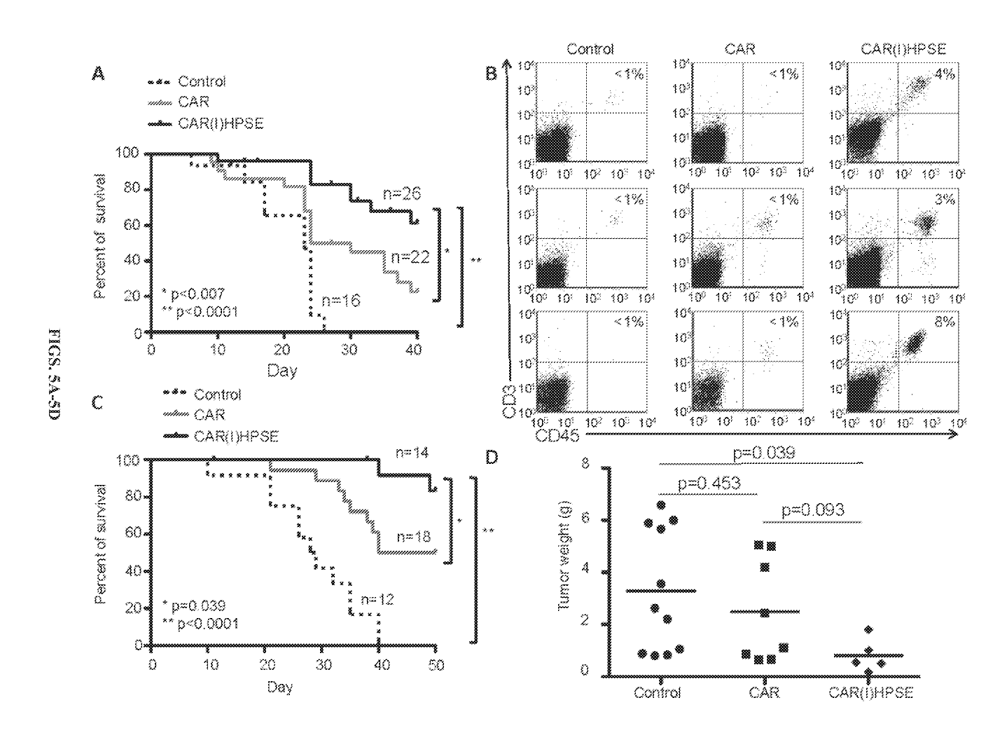

To validate the findings in vivo, a xenograft model of NB was established by implanting NOG/SCID/γ_{c}^{-/-} mice i.p. with two different cell line (CHLA-255 and LAN-1) in the presence of Matrigel to allow the formation of a complex and structured tumor. After 10 days, mice received either control LTE-T or CAR⁺ or CAR(I)HPSE⁺ LTE-T i.p. As shown in FIG. 5A, mice implanted with CHLA-255 and treated with CAR(I)HPSE⁺ LTE-T had a significantly improved day 40 survival as compared to mice treated with control LTE-T (p<0.001) or CAR⁺ LTE-T (p<0.007). At 40 days, surviving mice from each treatment group were euthanized and assessed for the presence of macroscopic tumors. Only 2 of 7 (29%) mice alive and infused with CAR⁺ LTE-T were tumor free, while 8 of 17 (47%) mice alive and infused with CAR(I)HPSE⁺ LTE-T had no evidence of tumor. In another set of experiments, mice were euthanized on day 12-14 after T-cell infusion to measure T-cell infiltration at the tumor site. The tumors of mice infused with CAR(I)HPSE⁺ LTE-T had greater infiltration of T cells (4.6%±2.4%), as compared to tumors collected from mice treated with control (0.6%±0.5; p=0.029) or CAR⁺ LTE-T (0.1%±0.1; p=0.043) (FIG. 5B). Similar results were obtained in mice engrafted with the tumor cell line LAN-1 (FIG. 5C). Mice infused with CAR(I)HPSE⁺ LTE-T had a significantly improved day 40 survival as compared to mice treated with control LTE-T (p<0.0001) or CAR⁺ LTE-T (p<0.039). Tumors collected from euthanized mice also showed a significant reduction in weight when mice were infused with CAR(I)HPSE⁺ LTE-T as compared to control (0.8 g±0.6 g vs. 3.3 g±2.4 g) (p=0.039), and a trend when compared to mice infused with CAR⁺ LTE-T (0.8 g±0.6 g vs. 2.5 g±2 g) (p=0.093) (FIG. 5D).

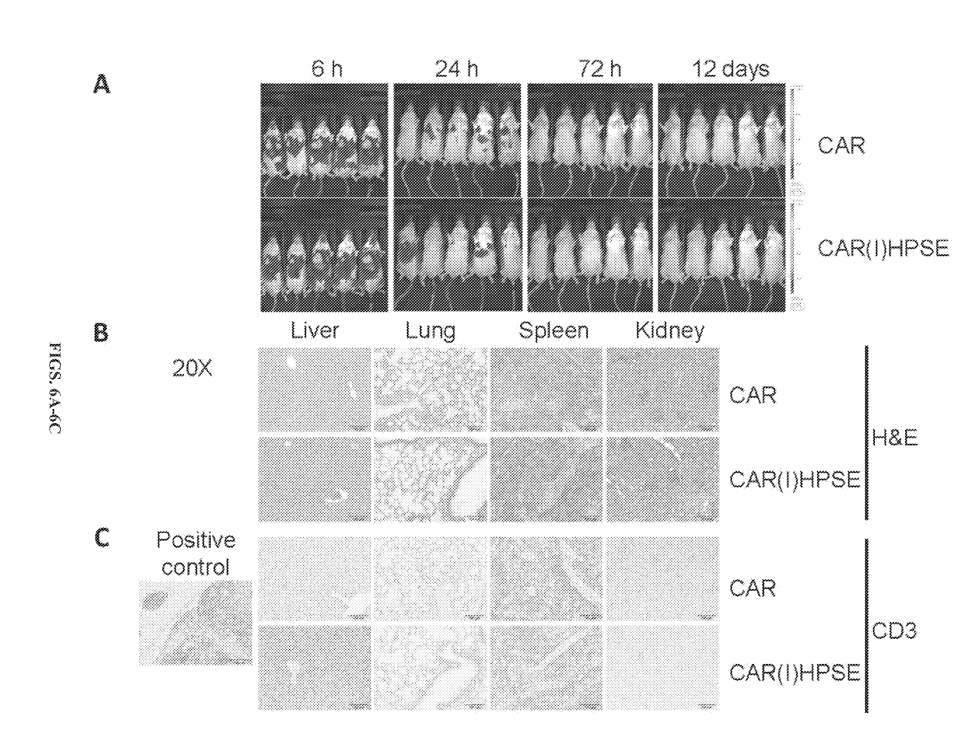

It was evaluated whether the forced expression of HPSE by T cells affects their biodistribution in vivo. For these studies, CAR(I)HPSE⁺ and CAR⁺ LTE-T were labelled with the vector encoding eGFP.FFluc and then infused via tail injection. T-cell biodistribution was evaluated by in vivo imaging at different time points after T cell infusion and did not show significant differences between the two groups of mice (FIG. 6).

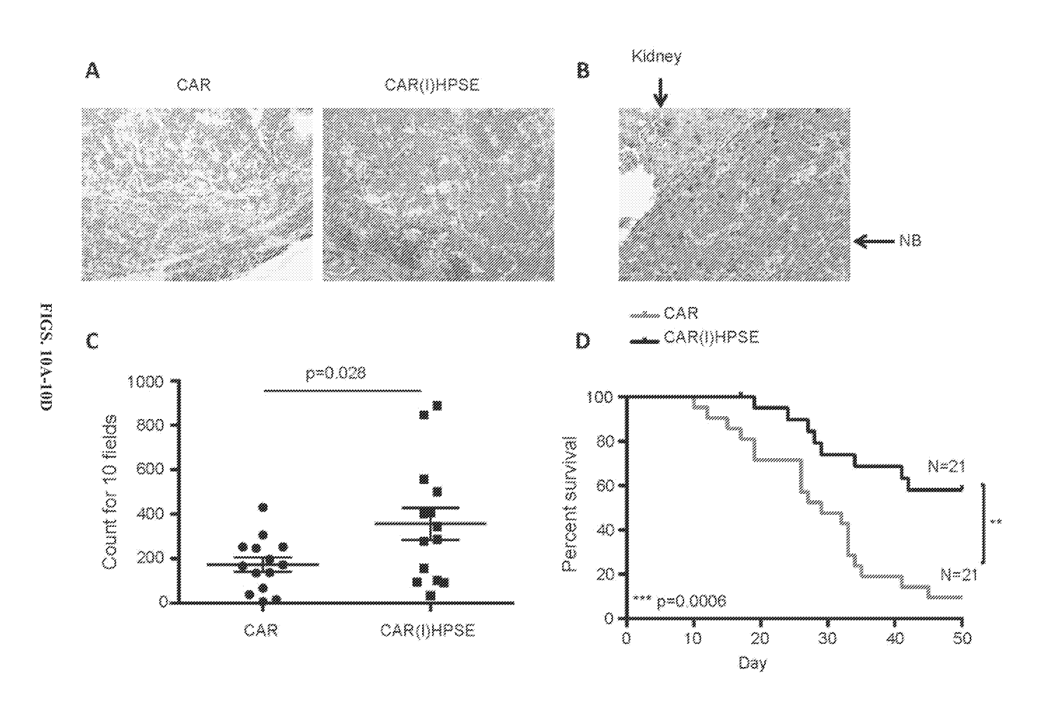

Because NB cell lines require Matrigel™ to form complex and structured tumors when infused i.p., the relevance of the proposed approach was validated in promoting T-cell infiltration of the tumor in a second NOG/SCID/γ_{c}^{-/-} model, in which CHLA-255 tumor cells labeled with Firefly luciferase are implanted in the kidney and develop solid tumors without the need for Matrigel™. Tumor sections from mice infused intravenously with CAR(I)HPSE⁺ LTE-T showed enhanced infiltration of CD3⁺ T cells compared to CAR⁺ LTE-T (357±72 and 173±32, respectively; p=0.028) (FIG. 10A,B,C). Long-term observation of infused mice also showed improved survival of mice infused with CAR(I)HPSE⁺ LTE-T by day 50 (p<0.005) (FIG. 10D).

To rule out concerns about non specific infiltration of normal tissues, such as lung or liver, by LTE-T with restored HPSE expression, in vivo T-cell bio-distribution was evaluated. For these studies, CAR(I)HPSE⁺ and CAR⁺ LTE-T were therefore labelled with the vector encoding GFP.FFluc and then infused via tail injection. T-cell bio-distribution evaluated by in vivo imaging and immunoistochemestry at different time points after T-cell inoculation did not show significant differences between the two groups of mice, indicating no preferential accumulation in lung or liver of LTE-T with re-stored expression of HPSE (FIG. 6).

### Example 3

### Summary of Certain Embodiments

The data show that the prolonged ex vivo culture required to generate tumor antigen-specific T cells for treatment of cancer impairs their production of HPSE, a key player in the degradation of the HSPGs that compose the tumor ECM. Lack of HPSE limits tumor-directed T cell migration through the ECM, impeding access to the tumor cells and reducing their ability to eliminate solid tumors. Forced expression of HPSE by gene transfer restores the capacity of CAR-redirected T cells to degrade HSPGs and enhances their antitumor effects in a NB model.

The capacity of T lymphocytes to extravasate through blood vessels to the tumor site is crucial for their antitumor function. While FT and BAT-L show detectable protein expression of the active 50 kDa form, LTE-T generated according to protocols currently used to manufacture T-cell lines for adoptive immunotherapy are HPSE deficient. The experiments show that HPSE mRNA is immediately down-regulated after T-cell activation, while HPSE-specific enzymatic activity increases within the first 72 hours post T-cell activation in the culture media. These observations are in line with previous studies showing that preformed HPSE is stored in an intracellular compartment and released as an early event in response to the activation of T cells (Bartlett, et al., 1995). Importantly, rapid transition from the inactive to the active form of HPSE and its release is a physiologic property of central-memory and effector-memory T cells upon TCR triggering. The analysis of T cells expanded ex vivo also shows that these cells both lack HPSE mRNA expression and enzymatic activity, and that neither transcription nor production of HPSE are restored when LTE-T are rested and then reactivated by TCR stimulation.

While the data show that lack of HPSE directly reduces the ability of LTE-T to degrade the HSPGs and thus hampers their antitumor activity in the presence of the ECM, it is also important to note that cleavage of HS chains releases preformed stored chemokines into the stroma (Gallagher, 2001; Iozzo, 1998). Since these chemokines guide the migration of T cells towards their target cells within the tumor microenvironment, the lack of HPSE may further indirectly compromise the antitumor effects of T cells by reducing their migration.

Re-expression of HPSE in LTE-T by gene transfer restores their physiologic capacity to degrade the ECM, without compromising the effector function. HPSE can readily be combined with a tumor directed CAR in a single vector, allowing the simultaneous acquisition of antitumor properties in addition to the restored degradation of the ECM. This leads in vivo to an increased numeric infiltration of T cells co-expressing CAR and HPSE within the tumor environment reflecting their restored capacity to degrade the ECM of the tumor stroma. The approach described herein allows the HPSE⁺CAR⁺ LTE-T to receive co-stimulation following CAR engagement through the inclusion of the CD28 and OX40 co-stimulatory endodomains within the CAR (Pule, et al., 2005). CAR-T cells lacking HPSE, however, do not engage the tumor cells and so can receive neither antigen-mediated stimulation nor co-stimulation so that the overall effect determined by the lack of HPSE is an increase in tumor growth in mice.

Under physiological conditions, HPSE expression by T cells is tightly regulated to avoid tissue damage from T-cell extravasation into non-pathologic tissues. In embodiments of the disclosure, HPSE is only expressed in CAR-T cells, and because antigen-specificity should drive accumulation of T cells preferentially in tissues with high antigen content (Marelli-Berg, et al., 2010), non-specific tissue infiltration should be limited; certainly, no changes in biodistribution, tissue infiltration or toxicity in mice infused with HSPE⁺CAR⁺ LTE-T.

In conclusion, the inventors have identified a specific deficit of HPSE in tumor-specific LTE-T that limits their antitumor activity and that can be overcome by forced expression of the enzyme. Employing this strategy significantly enhances the activity of tumor-directed T cells in patients with solid tumors.

### Example 4

### Exemplary Material and Methods

Cell Lines.

The cell lines 293T (human embryonal kidney), DU-145 (human prostate cancer), A549 (human lung epithelial carcinoma) and CHLA-255 (NB) were cultured in IMDM (Gibco, Invitrogen™, Carlsbad, Calif., USA) supplemented with 10% fetal bovine serum (FBS, Hyclone, Thermo Scientific, Pittsburgh, Pa., USA) and 2 mM GlutaMax (Invitrogen, Carlsbad, Calif.). The cell lines MCF-7 (breast cancer), Raji (Burkitt's lymphoma), K562 (eritromyeloblastoid leukemia) and LAN1 (NB) were cultured in RPMI1640 (HyClone) supplemented with 10% FBS and 2 mM GlutaMax. Cells were maintained in a humidified atmosphere containing 5% CO₂ at 37° C.

Isolation and Culture of Primary Human T Lymphocytes.

Peripheral blood mononuclear cells (PBMC) were isolated from buffy coats of healthy donors (Gulf Coast Regional Blood Center, Houston, Tex., USA) using Ficoll-Paque (Amersham Biosciences, Piscataway, N.J.). Monocytes were obtained from PBMC by positive magnetic selection with CD14 microbeads (Miltenyi Biotec, Auburn, Calif., USA). CD8⁺ and CD4⁺ T cells were obtained from PBMC by negative magnetic selection using specific microbeads (Miltenyi). CD8⁺ and CD4⁺ T cells were obtained from PBMC by negative magnetic selection using specific microbeads (Miltenyi). In selected experiments, central-memory cells (CD45RO⁺CD62L⁺) and effector-memory cells (CD45RO⁺CD62L⁻) were also separated from PBMC by positive magnetic selection (Miltenyi). T lymphocytes were activated with immobilized anti-CD3 (OKT3) (1 μg/ml) and anti-CD28 (Becton Dickinson Biosciences, Franklin Lakes, N.J., USA) (1 μg/ml) antibodies (Abs) and then expanded in complete medium containing 45% RPMI1640 and 45% Click's medium (Irvine Scientific, Santa Ana, Calif., USA) supplemented with 10% FBS and 2 mM GlutaMAX. Cells were fed twice a week with recombinant interleukin-2 (IL-2) (50 U/mL) (Chiron Therapeutics, Emeryville, Calif., USA), as previously described (Savoldo, et al., 2007).

Invasion Assay.

The capacity of each cell subset to degrade the ECM was examined in vitro using the BioCoat™ Matrigel™ Invasion assay (BD Biosciences) following the manufacturer's instructions. Briefly, cells maintained in serum-free medium for 18 hours were seeded (2.5×10⁵ cells/chamber) in the upper chamber/insert. Media supplemented with 10% FBS was added to the lower compartment to act as a chemo attractant. After 24 hours, cells in the lower chamber were counted by trypan blue exclusion. All experiments were performed in duplicate. Data are expressed as the percentage of invasion through the Matrigel and the membrane relative to the migration through the control membrane (8 μm polyethylene terephthalate membrane pores). The percentage of invasion was calculated as follows: (mean of cells invading through the Matrigel chamber membrane/mean of cells migrating through the control insert membrane)×100. In specific experiments, the invasion and antitumor activity of T lymphocytes were simultaneously evaluated. Briefly, the BioCoat™ Matrigel™ Invasion assay was used, with plated LAN1/GFP⁺ or CHLA 255/GFP⁺ cells (14×10⁴) in the bottom of a 24 well plate and T cells (2.5×10⁵ cells) in the upper chamber/insert. The chamber and insert were removed 24 hours later, and after three further days of culture, cells were collected from the lower chamber quantified by flow cytometry to identify tumor cells and T cells, respectively.

Western Blot.

CD4⁺ and CD8⁺ T cells were collected at different time points after activation with OKT3/CD28 Abs. Proteins were extracted from 5×10⁶ cells, using RIPA lysing buffer (Cell Signaling Technology®, Danvers, Mass., USA) supplemented with a protease inhibitor cocktail (Sigma-Aldrich, St. Louis, Mo., USA). Fifty μg of proteins were resolved by SDS-PAGE, transferred to polyvinylidene difluoride membranes (Bio-Rad, Hercules, Calif.) and blocked with 5% (W/V) non-fat dry milk in Tris Buffer Saline (TBS) with 0.1% (V/V) Tween-20 before being probed with the appropriate Abs. The Abs and dilutions used in these experiments were as follows: mouse anti-human HPA1 (1:100, clone HP130) (InSight Biopharmaceuticals Ltd, Rehovot, Israel) that recognizes both the 65 kDa precursor and the 50 kDa active form of HPSE-1, rabbit anti-human HPA1 polyclonal (1:4000 Cedarlane, Burlington, N.C., USA) and mouse anti-human β-actin (1:10000, clone C4) (Santa Cruz Biotechnology, Santa Cruz, Calif., USA). Blots were washed with TBS containing 0.1% (V/V) Tween-20 and then stained with horseradish peroxidase conjugated secondary Abs which were diluted in blocking solution (1:5000, goat anti-mouse sc-2005 and goat anti-rabbit sc-2004) (Santa Cruz). Blots were then incubated with SuperSignal West Femto Maximum Sensitivity Substrate (Thermo Scientific).

Immunofluorescence.

Adherent cells (1×10⁵ cells/well) were grown on Lab-Tek II chamber slide w/cover (Nalge Nunc Intl, Roskilde, Denmark) while non-adherent cells (3.5×10⁵ cells) were cytospun onto microscope slides. Cells were fixed with 4% paraformaldehyde (v/v). After permeabilization with 0.1% Triton X-100 (v/v), cells were incubated with 5% goat serum (Cell Signaling Tecnology®) and 1% BSA to block non-specific binding and then stained with the primary antibody against human HPSE1 (HPA1, clone HP130) (InSight Biopharmaceuticals Ltd) (1:100 dilution at room temperature for 2 hours). Cells were then probed with Alexa Fluor 555 goat anti-mouse secondary antibody (1:500 dilution at room temperature for 2 hours) (Cell Signaling Technology®, Danvers, Mass., USA). Fluorescent signals were detected using a fluorescence microscope (Olympus IX70, Leeds Instruments Inc, Irving, Tex., USA). DAPI was used as nuclear staining.

RNA Isolation and Quantitative Real-Time PCR (qRT-PCR).

CD4⁺ and CD8⁺ T cells were collected at different time points after activation with OKT3/CD28 Abs. For the qRT-PCR, 100 ng of total RNA were used to prepare cDNA (TaqMan One Step PCR Master Mix Reagents Kit) (Applied Biosystem, Carlsbad, Calif., USA). Specific primers and probes for HPSE were used (Applied Biosystem) (HPSE: Hs00935036_m1). The difference in cycle threshold values (ΔCT) of HPSE was normalized to the ΔCT of GAPDH (Glyceraldehude-3-phospate dehydrogenase, Hs99999905_m1), and the fold-change in expression was expressed relative to CD14⁺ cells, considered as a positive control.

Enzyme-Linked Immunosorbent Assay (ELISA).

Cytokine release by T cells in response to stimulation with GD2⁺ LAN1 cells was analyzed using IFNγ and IL-2 specific ELISAs (R&D Systems, Minneapolis, Minn., USA). HPSE activity was measured using a heparan sulfate (HS) degrading enzyme assay kit (Takara Bio Inc, Otsu, Shiga, Japan). Briefly, biotinylated HS was used as a substrate for the enzyme. The non-degraded substrate bound to fibroblast growth factor was then detected with avidin-peroxidase, and the absorbance measured at 450 nm. HPSE activity was determined as the inverse of decrease in absorbance as previously described (Roy, et al., 2005; Zhang, et al., 2010). T cell and tumor cell supernatants were analysed in triplicate. Supernatants were incubated with biotinylated HS at 37° C. for 75 minutes and HPSE-1 activity was determined by an ELISA-type assay. Color was developed using the specific substrate and plates were read at 450 nm using a microplate reader (ELx808iu, Bio-Tek Instruments). As described previously for Western blot and qRT-PCR, supernatants were collected from CD4⁺ and CD8⁺ T cells at different time points after activation with OKT3/CD28 Abs. On days 4 and 14 after activation, cells were collected, counted, washed and re-plated in fresh media.

Retroviral Constructs, Transient Transfection and Transduction of T Lymphocytes.

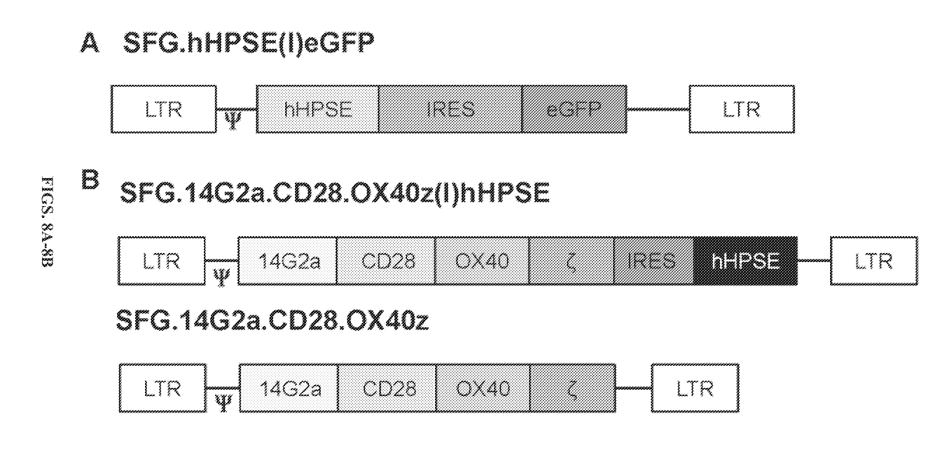

HPSE cDNA (accession number NM-006665) was cloned into the SFG retroviral backbone that also encodes the eGFP (SFG.HPSE(I)eGFP) (FIG. 8). The construct for the GD2-specific CAR containing the CD28, OX40 and ζ endodomains was previously described (SFG.CAR) (Pule, et al., 2005). The inventors then generated an exemplary bicistronic vector to co-express the HPSE and CAR-GD2 using an IRES (SFG.CAR(I)HPSE) (FIG. 8). The retroviral vector encoding the fusion protein eGFP-firefly luciferase (eGFP.FFLuc) for in vivo imaging of T cells was previously described (Vera, et al., 2006). To produce the retroviral supernatant, 293T cells were co-transfected with retroviral vectors, Peq-Pam plasmid encoding the MoMLV gag-pol, and the RDF plasmid encoding the RD114 envelope, as previously described (Vera, et al., 2006). A specific inhibitor of HPSE, SST0001 (a chemically modified heparin ¹⁰⁰Na,Ro-H) (3 μg/ml) (Vlodaysky, et al., 2007; Naggi, et al., 2005), was added to the media during the virus preparation to increase its titer. Activated T lymphocytes were then transduced with retroviral supernatants using retronectin-coated plates (Takara Bio Inc). After removal from the retronectin plates, T-cell lines were maintained in complete T-cell medium in a humidified atmosphere containing 5% CO₂ at 37° C. in the presence of IL-2 (50 U/mL) for 2 weeks.

Flow Cytometry.

The inventors used the following exemplary Abs: CD45, CD56, CD8, CD4, and CD3 (all from Becton Dickinson, San Jose, Calif.) conjugated with FITC, PE, PerCP or APC fluorochromes. The inventors included control samples labelled with appropriate isotype-matched Abs in each experiment. The expression of GD2-specific CAR in T lymphocytes was detected using a specific anti-idiotype antibody (1A7) (Rossig, et al., 2002). Samples were analyzed with a BD FACScalibur system equipped with the filter set for quadruple fluorescence signals and the CellQuest software (BD Biosciences). For each sample the inventors analyzed a minimum of 10,000 events.

Chromium-Release Assay.

The cytotoxic activity of T cells was evaluated using a standard 6-hour ⁵¹Cr-release assay, as previously described (Savoldo, et al., 2002). Target cells were incubated in medium alone or in 1% Triton X-100 (Sigma-Aldrich) to determine spontaneous and maximum ⁵¹Cr release, respectively. The mean percentage of specific lysis of triplicate wells was calculated as follows: [(test counts−spontaneous counts)/(maximum counts−spontaneous counts)]×100. The target cells tested included LAN1, CHLA 255 and Raji.

Xenogenic SCID Mouse Model.

The inventors used a previously-described SCID mouse model (Savoldo, et al., 2007; Quintarelli, et al., 2007), to assess the in vivo antitumor effect of control and T cells transduced with either the SFG.CAR or the SFG.CAR(I)HPSE retroviral vectors. Mouse experiments were performed in accordance with Baylor College of Medicine's Animal Husbandry guidelines. Eight-10 week old NOG/SCID/γ_{c}^{-/-} mice (Jackson Lab, Bar Harbor, Me.) were injected intraperitoneally (i.p.) with CHLA 255 cells (2.5×10⁶) resuspended in Matrigel (BD Biosciences). Ten-twelve days after tumor inoculation, T cells were injected i.p. (20×10⁶ cells/mouse). Mice were euthanized when signs of discomfort were detected. For the in vivo biodistribution of T cells, 5×10⁶ T cells/mouse labelled with the eGFP.FFluc vector were infused via tail injection. For in vivo imaging the Xenogen-IVIS Imaging System was used as previously described (Vera, et al., 2006).

Statistics.

Unless otherwise noted, data are summarized as mean±standard deviation (SD). Student t-test was used to determine the statistical significant differences between samples, with P value <0.05 indicating a significant difference. When multiple comparison analyses were required, statistical significance was evaluated by a repeated measures ANOVA followed by a Newman-Keuls or Log-rank (Mantel Cox) test for multiple comparisons. The survival data of the mice were analysed using the Kaplan-Meier survival curve.

### REFERENCES

All patents and publications cited herein are hereby incorporated by reference in their entirety herein.
Bartlett, et al., Immunol. Cell Biol. 73:113-124, 1995.
Bernfield, et al., Annu. Rev. Biochem. 68:729-777, 1999.
de Mestre, et al., J. Leukoc. Biol. 82:1289-1300, 2007.
Fridman, et al., J. Cell Physiol 130:85-92, 1987.
Gallagher, J. Clin. Invest 108:357-361, 2001.
Iozzo, Annu. Rev. Biochem. 67:609-652, 1998.
Joyner, et al., Nature. 338:153-156, 1989.
Kalos, et al., Sci. Transl. Med. 3:95ra73, 2011.
Mansour, et al., Nature. 336:348-352, 1988.
Marelli-Berg, et al., Immunology 130:158-165, 2010.
Morgan, et al., Science 314:126-129, 2006.
Muller, Trends Immunol. 24:327-334, 2003.
Naggi, et al., J. Biol. Chem. 280:12103-12113, 2005.
Naparstek, et al., Nature 310:241-244, 1984.
Parish, Nat. Rev. Immunol. 6:633-643, 2006.
Pule, et al, Nat. Med. 14:1264-1270, 2008.
Pule, et al., Mol. Ther. 12:933-941, 2005.
Quintarelli, et al., Blood 110:2793-2802, 2007.
Rooney, et al., Lancet 345:9-13, 1995.
Rossig, et al., Blood 99:2009-2016, 2002.
Roy, et al., Neoplasia. 7:253-262, 2005.
Savoldo, et al., Blood 100:4059-4066, 2002.
Savoldo, et al., Blood 110:2620-2630, 2007.
Savoldo, et al., J. Clin. Invest 121:1822-1826, 2011.
Thomas & Capecchi, Cell. 51:503-512, 1987.
Vera, et al., Blood 108:3890-3897, 2006.
Vlodaysky, et al., Curr. Pharm. Des 13:2057-2073, 2007.
Vlodaysky, et al., Invasion Metastasis 12:112-127, 1992.
Yadav, et al., Thromb. Haemost. 90:598-606, 2003.
Yurchenco & Schittny, FASEB J. 4:1577-1590, 1990.
Zhang, et al., Mol. Cancer Res. 8:278-290, 2010.
Zou, Nat. Rev. Cancer 5:263-274, 2005.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An ex vivo cultured cell, comprising recombinant expression of heparanase, wherein there is no expression of endogenous heparanase in the cell or wherein existing expression of heparanase is overexpressed upon recombinant expression of heparanase.

2. The cell of claim 1, wherein the cell is a T cell, NK cell, or NKT cell.

3. The cell of claim 1, wherein the cell is an ex vivo expanded T cell.

4. The cell of claim 1, wherein the cell is a tumor antigen-specific T cell.

5. The cell of claim 1, wherein the cell is a chimeric antigen receptor (CAR)-specific T cell.

6. A method of improving efficacy of cell therapy, comprising the step of modifying cells for the therapy to express heparanase.

7. The method of claim 6, wherein the cells lack endogenous heparanase expression and the modifying step restores heparanase expression.

8. The method of claim 6, wherein the cells have endogenous heparanase expression and the heparanase is overexpressed.

9. The method of claim 6, wherein the cells are tumor antigen-specific T cells.

10. The method of claim 6, wherein the cells are CAR-specific T cells.

11. The method of claim 6, wherein the cell therapy is for cancer.

12. The method of claim 6, wherein the cell therapy is for a solid tumor.

13. A method of treating cancer in an individual, comprising the step of delivering a therapeutically effective amount of therapeutic cells to the individual, wherein the cells are ex vivo cultured cells that recombinantly express heparanase.

14. The method of claim 13, wherein the cells:
1) lack endogenous heparanase expression; or
2) have endogenous heparanase expression and the recombinantly expressed heparanase is overexpressed.

15. The method of claim 13, wherein the cancer comprises solid tumor.

16. The method of claim 13, wherein the cells are tumor antigen-specific T cells.

17. The method of claim 13, wherein the cells are CAR-specific T cells.
